(19) United States
(12) Patent Application Publication
(10) Pub. No.: US 2024/0044854 A1
(43) Pub. Date: Feb. 8, 2024

(54) Measurement, Reporting, And Verification (MRV) For Ocean Carbon Dioxide Removal Systems

(71) Applicant: Ebb Carbon, Inc., San Carlos, CA (US)

(72) Inventors: Matthew Eisaman, Port Jefferson, NY (US); Todd Pelman, Moss Beach, CA (US); Jeremy Loretz, Palo Alto, CA (US)

(73) Assignee: Ebb Carbon, Inc., San Carlos, CA (US)

(21) Appl. No.: 18/228,399

(22) Filed: Jul. 31, 2023

Related U.S. Application Data
(60) Provisional application No. 63/370,326, filed on Aug. 3, 2022.

(51) Int. Cl. G01N 31/22; G01N 33/18; B01D 53/14; B01D 53/62

(52) U.S. Cl. CPC G01N 31/223; G01N 33/18; B01D 53/1475; B01D 53/62; B01D 2252/1035; B01D 2257/504; B01D 2251/604

(57) ABSTRACT

MRV for an ocean CDR system is achieved by varying the release/delivery of base substance into ocean seawater such that the base substance propagates as a series of release batch wavefronts along a dispersion path. A release frequency, which controls a timing of the release batch wavefronts, is selected to coincide with a non-natural frequency (e.g., a frequency exhibiting quiet/weak power spectra in a natural seawater chemistry variation power spectrum). Time-based seawater carbonate chemistry measurement data, which is collected by ocean-based sensors disposed in the base substance's dispersion path during base substance release, records both human-induced contributions caused by the release batch wavefronts and natural seawater chemistry variations. The time-based sensor data is processed using frequency-domain techniques to generate a power spectrum in which human-induced contributions at the non-natural frequency can be distinguished from natural variation contributions, thereby facilitating verification of the system's contribution to atmospheric CO₂ reduction.

### PRIORITY APPLICATION

The present application claims priority to U.S. Provisional Patent Application 63/370,326 entitled “MEASUREMENT, REPORTING, AND VERIFICATION (MRV) FOR ELECTROCHEMICAL OCEAN ALKALINITYENHANCEMENT SYSTEMS”, filed by Eisaman et al. on Aug. 3, 2022.

### FIELD OF THE INVENTION

The systems and methods described herein relate to ocean Carbon Dioxide Removal systems that utilize various base generating devices to both reduce atmospheric carbon dioxide (CO₂) and mitigate ocean acidification.

### BACKGROUND OF THE INVENTION

As humans burn more and more fossil fuels, the resulting increased carbon dioxide (CO₂) concentration in Earth's atmosphere causes both climate change and ocean acidification. The increased atmospheric concentrations of CO₂ and other greenhouse gasses (e.g., methane) produces climate change by trapping heat close to earth's surface, thereby increasing both air and sea temperatures. Because earth's oceans absorb about 25% of atmospheric CO₂, and because the absorbed CO₂ dissolves to form carbonic acid that remains trapped in the seawater, the increased atmospheric CO₂ concentration caused by burning fossil fuels also produces ocean acidification by way of increasing the amount of CO₂ gas dissolved in the ocean.

Both climate change and ocean acidification pose significant threats to humans. Climate change in the form of increased global average temperatures can produce several dangerous effects such as the loss of polar ice and corresponding increased sea levels, disease, wildfires and stronger storms and hurricanes. Ocean acidification changes the ocean chemistry that most marine organisms rely on. One concern with ocean acidification is that the decreased seawater pH can lead to the decreased survival of shellfish and other aquatic life having calcium carbonate shells, as well as some other physiological challenges for marine organisms.

To avoid dangerous climate change, the international Paris Agreement aims to limit the increase in global average temperature to no more than 1.5° C. to 2° C. above the temperatures of the pre-industrial era. Global average temperatures have already increased by between 0.8° C. and 1.2° C. The Intergovernmental Panel on Climate Change (IPCC) estimates that a ‘carbon budget’ of about 500 GtCO₂ (billion tons of carbon dioxide), which corresponds to about ten years at current emission rates, provides a 66% chance of limiting climate change to 1.5° C.

In addition to cutting CO₂ emissions by curtailing the use of fossil fuels, climate models predict that a significant deployment of Negative Emissions Technologies (NETs) will be needed to avoid catastrophic ocean acidification and global warming beyond 1.5° C. (see “Biophysical and economic limits to negative CO₂ emissions”, Smith P. et al., Nat. Clim. Chang. 2016; 6: 42-50). Current atmospheric CO₂ and other greenhouse gas concentrations are already at dangerous levels, so even a drastic reduction in greenhouse gas emissions would merely curtail further increases, not reduce atmospheric greenhouse gas concentrations to safe levels. Moreover, the reduction or elimination of certain greenhouse gas sources (e.g., emissions from long-distance airliners) would be extremely disruptive and/or expensive and are therefore unlikely to occur soon.

Therefore, there is a need to supplement emission reductions with the deployment of NETs, which are systems/processes that serve to reduce existing atmospheric greenhouse gas concentrations by, for example, capturing/removing CO₂ from the air and sequestering it for at least 1,000 years. The need for NETs may be explained using a bathtub analogy in which atmospheric CO₂ is represented by water contained in a bathtub, ongoing CO₂ emissions are represented by water flowing into the tub, and NETs are represented by processes that control water outflow through the tub's drain. In this analogy, reduced CO₂ emission rates are represented by partially turning off the water inflow tap—the slower inflow rate provides more time before the tub fills, but the tub's water level will continue to rise and eventually overflow. Using this analogy, although reducing CO₂ emissions may slow the increase of greenhouse gas in the atmosphere, critical concentration levels will eventually be reached unless NETs are implemented that can offset the reduced CO₂ emission level (i.e., remove atmospheric CO₂ at the same rate it is being emitted). Moreover, because greenhouse gas concentrations are already at dangerous levels (i.e., the tub is already dangerously full), there is an urgent need for NETs that are capable of significantly reducing atmospheric CO₂ faster than it is being emitted to achieve safe atmospheric concentration levels (i.e., outflow from the tub's drain must be greater than the reduced inflow from the tap to reduce the tub's water to a safe level).

NETs can be broadly characterized as Direct Air Capture (DAC) approaches and ocean Carbon Dioxide Removal (ocean CDR) approaches. DAC approaches utilize natural (e.g., reforestation) and technology-based methods to extract CO₂ directly from the atmosphere. Ocean CDR approaches (a subset of which are referred to as “ocean alkalinity enhancement (OAE)” approaches and a separate subset of which are sometimes referred to as either “direct ocean capture (DOC)” or “indirect ocean capture (IOC)” approaches) utilize various natural and/or technological processes to remove CO₂ from the atmosphere using the ocean. OAE approaches do this by generating and supplying an ocean alkalinity product (i.e., a base substance or alkaline solution) to ocean seawater, thereby increasing the ocean's ability to absorb atmospheric CO₂ and store it in the ocean as bicarbonate, a form of carbon storage that is stable for over 10,000 years. DOC/IOC approaches do this by generating an acid and a base, using the acid to shift all dissolved carbon in seawater to CO₂ gas, stripping the CO₂ gas from the seawater, and then restoring the lost alkalinity by adding the base to the seawater, resulting in the absorption by the seawater of an amount of CO₂ from the air equal to that stripped out in the first step.

Electrochemical Ocean Alkalinity Enhancement (electrochemical OAE) represents an especially promising ocean CDR approach that both reduces atmospheric CO₂ and mitigates ocean acidification by generating an ocean alkalinity product comprising an aqueous alkaline solution containing a fully dissolved base substance and, in some embodiments, a salt. Electrochemical OAE systems typically generate the required base substance using a bipolar electrodialysis device (BPED), which generally includes an ion exchange (IE) stack that utilizes an electrochemical process to convert salt supplied in a feedstock solution into the base substance and an acid substance. Other OAE systems utilize different approaches to generate ocean alkalinity products that are suitable for release into the ocean. For example, Mineral Ocean Alkalinity Enhancement (mineral OAE) approaches utilize crushed rock or mined magnesium hydroxide (Mg(OH)₂) to generate solid base substance particles that are then released into the ocean (i.e., the solid base substance particles entirely form or are included in the mineral OAE system's ocean alkalinity product). In other ocean CDR systems, a base substance (e.g., from crushed rock, mined Mg(OH)₂ or another source) may be dissolved in an aqueous solution or mixed with another solvent to produce a suitable ocean alkalinity product. All of these ocean CDR systems (e.g., mineral OAE systems, electrochemical OAE systems, DOC/IOC systems, or any other system that reduces atmospheric CO₂ by releasing base substance into the ocean as one the process steps) then supply their ocean alkalinity product to the ocean at a designated outfall location, whereby the base substance in the ocean alkalinity product gradually diffuses (disperses) into the surrounding seawater. In the case of OAE approaches, as the base substance diffuses into the surrounding ocean seawater it serves to directly reverse ocean acidification (i.e., by increasing the ocean seawater's alkalinity), and indirectly reduces atmospheric CO₂ (i.e., increasing the ocean seawater's alkalinity increases the ocean's ability to absorb/capture atmospheric CO₂). Because the generated base substance is fully dissolved in the ocean alkalinity product, the electrochemical OAE approach, or any approach using aqueous alkalinity, avoids problems associated with other ocean CDR approaches (e.g., dissolution kinetics issues that are associated with conventional mineral OAE approaches).

Although all ocean CDR systems show great potential in mankind's efforts to combat global warming, their widespread acceptance is hindered by Measurement, Reporting, and Verification (MRV) issues. MRV generally refers to the process by which entities (e.g., corporations, countries, etc.) track and report data on greenhouse gas (GHG) emissions, the implementation and impact of NET-based mitigation actions (e.g., atmospheric CO₂ capture/removal), and the finances used to support these NET-based mitigation actions (i.e., how efficient is each NET system in terms of unit cost of CO₂ capture/removal). In the context of ocean CDR approaches, MRV generally refers to measuring and verifying the amount of dispersed base substance into the ocean (which functions to capture/remove atmospheric CO₂ from the air over the ocean). In this context, ocean CDR approaches face several MRV-related problems arising from the natural periodic variations in the carbonate chemistry of the ocean and the relatively slow time scale (i.e., relative to the rate at which the alkalinity disperses throughout the ocean) of the equilibration of atmospheric CO₂ with the surface ocean, which makes it difficult to detect the change in carbonate chemistry (for example, the increase in dissolved inorganic carbon (DIC) in seawater) due to the human-induced ocean alkalinity enhancement (the signal) against the natural variation in carbonate chemistry and measurement uncertainty (the noise). That is, due to the relatively slow time scale of atmospheric CO₂ removal (i.e., capture in the ocean) in response to the addition of base substance (alkalinity) relative to the rate at which the alkalinity disperses throughout the ocean, direct measurement/verification of atmospheric CO₂ removal (CO₂ drawdown) that is direct result of an ocean CDR system's activity is, at best, difficult. Moreover, indirect measurement and verification of atmospheric CO₂ removal (e.g., by way of measuring changes to the ocean's pH to measure/verify the release of base substance) is also difficult due to signal-to-noise issues caused by natural variations in the ocean's pH (i.e., seawater pH measurement data collected at a given time includes significant noise due to unpredictable contribution caused by natural variations, thereby making it very difficult to detect and measure a relatively small contribution “signal” associated with the released base substance).

What is needed is a reliable method for measuring and verifying atmospheric CO₂ removal produced by base substances released from ocean CDR systems. In particular, what is needed is a method for indirectly measuring/verifying atmospheric CO₂ removal that reliably distinguishes changes to the ocean seawater carbonate chemistry caused by released base material from natural ocean seawater chemistry variations.

### SUMMARY OF THE INVENTION

A frequency-based base substance detection method for reliably verifying an ocean CDR system's contribution to atmospheric CO₂ removal is characterized by controlling the ocean CDR system to supply base substance to an ocean with a time waveform whose power spectrum contains significant contributions in at least one non-natural (quiet natural variation) frequency. In one embodiment, the ocean CDR system's control circuit controls a flow control device such that an ocean alkalinity product is supplied to the ocean at an outfall location as a series of discrete release batches (i.e., by alternately opening and closing the flow control device), whereby the higher base substance concentration supplied in each release batch propagates (disperses) through the seawater, thereby generating a series of wave-like base substance wavefronts that move away from outfall location along radial dispersion paths. During the base substance release, time-based seawater carbonate chemistry measurement data is collected by one or more stationary ocean-based sensors that are located in the base substance's dispersion path and configured to measure one or more seawater carbonate (carbon-related) parameter levels (e.g., pH, dissolved organic carbon (DIC), partial pressure of CO₂ (pCO₂) and total alkalinity (TA) of the seawater). The time-based seawater chemistry carbonate measurement data reflects changes (increases/decreases) to the one or more seawater carbon-related parameter levels that are caused by both (i) natural seawater carbonate chemistry variations (i.e., changes in pH, DIC, pCO₂ and/or TA caused by a wide range of naturally occurring processes and changes in ocean/atmospheric conditions) and (ii) human-induced changes in pH, DIC, pCO₂ and/or TA caused by the base substance wavefronts. The time-based seawater carbonate chemistry measurement data is then processed using frequency-domain techniques (e.g., Fourier transform conversion) to generate a seawater chemistry power spectrum including power spectra (contribution) values for a range of frequencies, where each power spectrum value indicates a relative contribution to seawater carbonate chemistry at a corresponding frequency. Note that, in the absence of human-induced changes to the one or more seawater carbon-related parameter levels caused by the dispersion of the base substance wavefronts (i.e., when the ocean CDR system is not in operation), the seawater chemistry power spectrum generated in this manner only includes power spectra values found in a natural seawater chemistry variation power spectrum. According to an aspect, the flow control device is controlled according to at least one selected release frequency that coincides with at least one non-natural (quiet natural variation) frequency (i.e., a frequency in the natural seawater chemistry variation power spectrum at which a zero or insignificant power spectra contribution is produced), and each released batch of ocean alkalinity product is generated with a sufficient amount of base material such that changes in seawater carbonate chemistry caused by the base substance wavefronts are detectable by the ocean-based sensors. By controlling the flow control device to release the series of released batches according to the selected release frequency, the resulting base substance wavefronts generated by the ocean CDR system produce a significant human-induced contribution (power spectra value) at the selected release frequency in the generated seawater chemistry variation power spectrum. Because the release frequency coincides with a non-natural (quiet natural variation) frequency, any significant power spectra value generated at the non-natural frequency in the seawater chemistry variation power spectrum provides clear evidence of a human-induced contribution, and therefore can be utilized to reliably verify the ocean CDR system's contribution to atmospheric CO₂ removal. Moreover, utilizing a quiet natural variation frequency as the selected release frequency effectively increases the signal-to-noise ratio of the human-induced contribution in the seawater chemistry variation power spectrum (i.e., by releasing base substance with a time waveform whose power spectrum contains the selected release frequency, the human-induced variation data “signal” is readily distinguished from the natural variation “noise”), thereby facilitating accurate measurement of the amount of base substance supplied by the ocean CDR system to the ocean. Accordingly, the frequency-based base substance detection method significantly increases the reliability of measuring and verifying an ocean CDR system's contribution to atmospheric CO₂ by generating human-induced contributions at one or more quiet natural variation frequencies (e.g., in comparison to ocean CDR systems that release alkalinity in a steady state manner or without regard to the frequencies of natural background variation).

In some embodiments the natural seawater chemistry variation data utilized to identify the selected release frequency is generated immediately before operation of an ocean CDR system is initiated. In such embodiments the same sensor network utilized to verify and measure the ocean CDR system's contribution to atmospheric CO₂ removal may be utilized to collect preliminary time-based seawater carbonate chemistry measurement data at locations along the base substance's expected dispersion path, then the preliminary time-based seawater carbonate chemistry measurement data may be processed as described above to generate a natural seawater carbonate chemistry variation power spectrum that can then be used to identify one or more non-natural (quiet) frequencies (i.e., frequencies having associated insignificant power spectra values). A benefit provided by identifying and selecting the release frequency(ies) subsequently used for base substance release is that this approach may provide the most accurate natural seawater chemistry variation data (i.e., because the time-based seawater carbonate chemistry data is obtained from the same locations and close in time to the data collected during verification/measurement operations). In other embodiments, the selected release frequency may be selected from previously generated natural seawater chemistry variation data (e.g., in cases where long-term analysis has concluded that the previously generated natural seawater chemistry variation data may be sufficiently accurate for purposes of selecting a release frequency).

In some embodiments an ocean CDR system utilizes preliminary seawater chemistry measurement data collected from one or more ocean-based sensors to maximize the signal-to-noise ratio in the seawater chemistry measurement data utilized to verify and measure ocean CDR system contributions by (i) identifying and implementing a suitable sensor network placement and/or (ii) identifying suitable base substance release frequencies. In one embodiment, the ocean-based sensor(s) is/are disposed in initial positions and utilized to establish a baseline seawater chemistry schedule (e.g., a model of time-based variations in seawater pH, DIC and/or TA parameter values due to naturally occurring changes in ocean/atmospheric conditions). The baseline seawater chemistry schedule is then utilized to (i) determine a suitable sensor network placement (e.g., optimize the location of the network's sensors for measuring spatial and temporal variations in seawater chemistry), and/or (ii) control the release of test amounts of base substance (i.e., such that each test amount is released at a preliminary release frequency that corresponds with an associated quiet natural frequency taken from the baseline seawater chemistry schedule). Once the sensor network placement is completed, one or more of the quiet natural frequencies that produce superior signal-to-noise ratios is/are selected as the release frequency utilized during subsequent ocean CDR system operations.

In another embodiment, a single sensor network may be utilized to measure and/or verify base substance releases (contributions) from two or more ocean CDR systems by coordinating the operations of the two or more ocean CDR systems such that each ocean CDR system supplies base substance into the same ocean region as a series of release batches but at unique (different) release frequencies. At least one sensor of the network is positioned within overlapping dispersion paths of the two or more ocean CDR systems such that time-based seawater chemistry measurement data collected by the sensor network is simultaneously influenced by the (first and second) series of release batches (i.e., by the base substance released from both ocean CDR systems). When this time-based seawater chemistry measurement data is subsequently processed using frequency-domain techniques to generate an associated seawater carbonate chemistry variation power spectrum, simultaneous verification of the contributions from both ocean CDR systems is provided by significant (non-zero) power spectra values appearing at the two (first and second) release frequencies. In some embodiments, one or more remote ocean-based sensors of the sensor network are positioned outside of expected dispersion paths and utilized to provide real-time baselining to enhance confidence in the accuracy of the processed data.

### BRIEF DESCRIPTION OF THE DRAWINGS

These and other features, aspects and advantages of the present invention will become better understood with regard to the following description, appended claims, and accompanying drawings, where:

FIG. 1 is a simplified diagram depicting an electrochemical OAE (ocean CDR) system configured to supply base substance to the ocean at a selected release frequency in accordance with an exemplary embodiment;

FIG. 2A is a timing diagram depicting an exemplary control signal generated in accordance with a selected release frequency that is utilized to control a flow control device of the electrochemical OAE system of FIG. 1;

FIG. 2B is a timing diagram depicting an exemplary time-base volumetric release of ocean alkalinity product (base substance) by the control device of the electrochemical OAE system of FIG. 1 in response to the control signal of FIG. 2A;

FIGS. 3A, 3B and 3C are diagrams depicting exemplary seawater pH time waveforms produced by the base substance wavefront of an exemplary released batch at three distances from an ocean alkalinity product outfall location;

FIG. 4 is a flow diagram depicting a frequency-based base substance detection method for verifying and measuring a contribution to atmospheric CO₂ removal by the electrochemical OAE system of FIG. 1 according to an exemplary embodiment;

FIG. 5 is a multilayer perspective diagram depicting the use of frequency-domain techniques to process time-based data according to a simplified example;

FIGS. 6A, 6B and 6C are diagrams depicting exemplary time-based seawater pH measurement data including both human-induced changes and natural seawater pH variations collected at three distances from an ocean alkalinity product outfall location;

FIGS. 7A, 7B and 7C are diagrams depicting exemplary power spectrums generated by applying frequency-domain techniques to the time-based seawater pH measurement data shown in FIGS. 6A, 6B and 6C, respectively;

FIG. 8 is a diagram depicting a BPED of the electrochemical OAE system of FIG. 1 according to an exemplary embodiment;

FIG. 9 is a flow diagram depicting a method for positioning and utilizing one or more ocean-based sensors to identify/select one or more non-natural release frequencies according to another embodiment;

FIG. 10 depicts a map showing a sensor network utilized to measure and/or verify base substance simultaneously released from two or more ocean CDR systems according to another embodiment; and

FIG. 11 is a graph showing exemplary time-based measurement data collected by the sensor network of FIG. 10; and

FIGS. 12A and 12B are graphs showing exemplary frequency-based representations generated by processing the time-based measurement data of FIG. 11 according to an embodiment.

### DETAILED DESCRIPTION OF THE DRAWINGS

The present invention relates to systems/methods for reliably measuring and verifying an ocean CDR system's contribution to the reduction of atmospheric CO₂. The following description is presented to enable one of ordinary skill in the art to make and use the invention as provided in the context of a particular application and its requirements. Various modifications to the preferred embodiment will be apparent to those with skill in the art, and the general principles defined herein may be applied to other embodiments. Therefore, the present invention is not intended to be limited to the particular embodiments shown and described but is to be accorded the widest scope consistent with the principles and novel features herein disclosed.

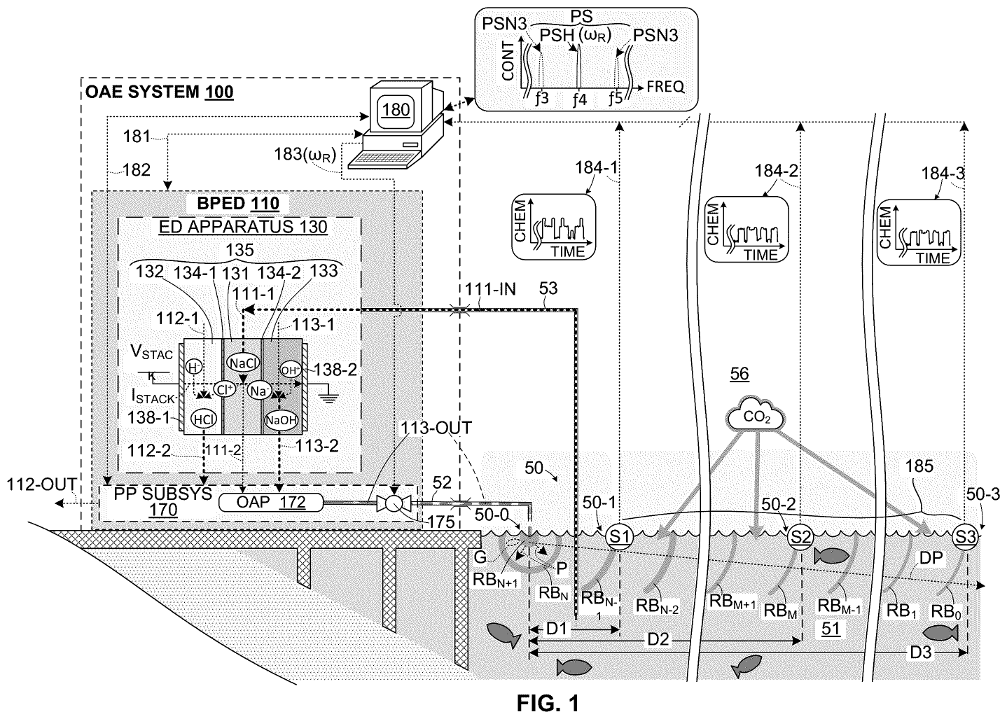

FIG. 1 shows an exemplary generalized electrochemical OAE (ocean CDR) system 100 that is configured to capture carbon dioxide (CO₂) from earth's atmosphere and mitigate ocean acidification by generating and supplying an ocean alkalinity product 113-OUT (i.e., an aqueous solution having a pH>8 and comprising salt and a dissolved base substance) to seawater 51 (ocean 50). In one embodiment, electrochemical OAE system 100 includes at least one BPED 110, which is configured to generate ocean alkalinity product 113-OUT, and a control circuit (controller) 180 that controls the generation and release of ocean alkalinity product 113-OUT into ocean 50. Electrochemical OAE system 100 may be located adjacent to ocean 50 and ocean alkalinity product 113-OUT may be transported (supplied) to ocean 50 by way of being pumped through an outflow transfer pipe 52 to an outfall location 50-0. Ocean alkalinity product 113-OUT is generated by BPED 110 such that the concentration of base substance in ocean alkalinity product 113-OUT is intentionally made higher than the concentration of base substance in seawater 51, whereby the base substance included with alkalinity product 113-OUT disperses by way of molecular diffusion into the surrounding seawater 51 (i.e., away from outfall location 50-0, for example, along dispersion path DP depicted by the dashed-line arrow in FIG. 1). Electrochemical OAE system 100 thus contributes to atmospheric CO₂ removal and reduces ocean acidification by increasing the alkalinity of seawater 51 (i.e., by way of the base substance supplied with ocean alkalinity product 113-OUT), thereby increasing the ability of ocean 50 to absorb/capture atmospheric CO₂ from atmospheric region 56 (i.e., the air over ocean 50). Of course, electrochemical OAE system 100 limits the supplied base substance (i.e., carefully controls the composition and outflow rate/quantity of ocean alkalinity product 113-OUT) to prevent harm to sea life that can be caused by dangerously high seawater pH levels near outfall location 50-0.

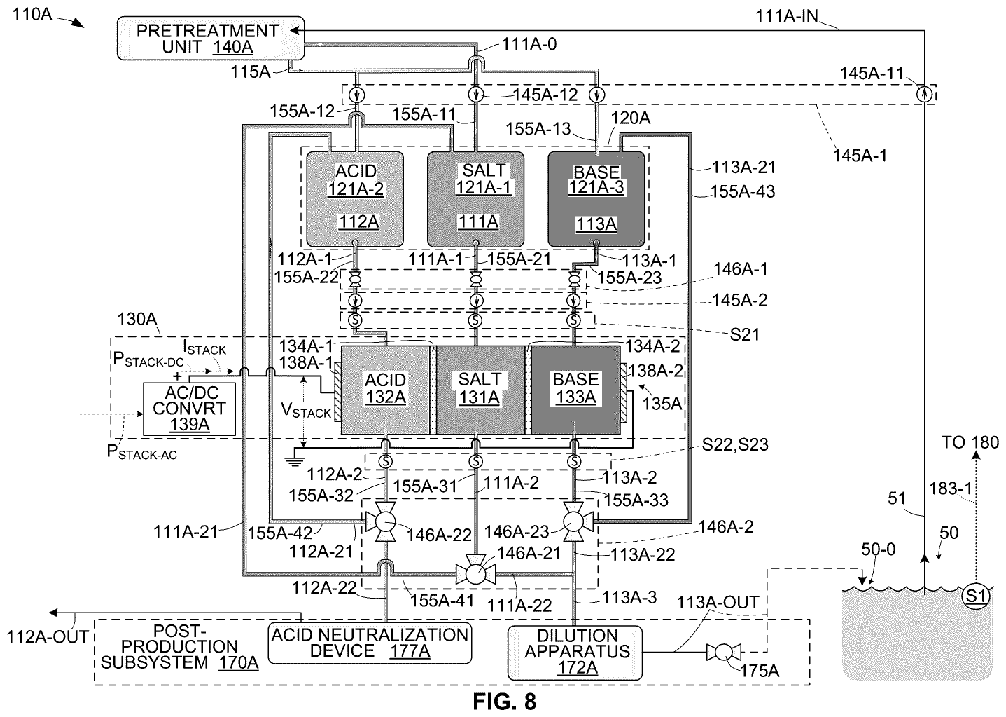

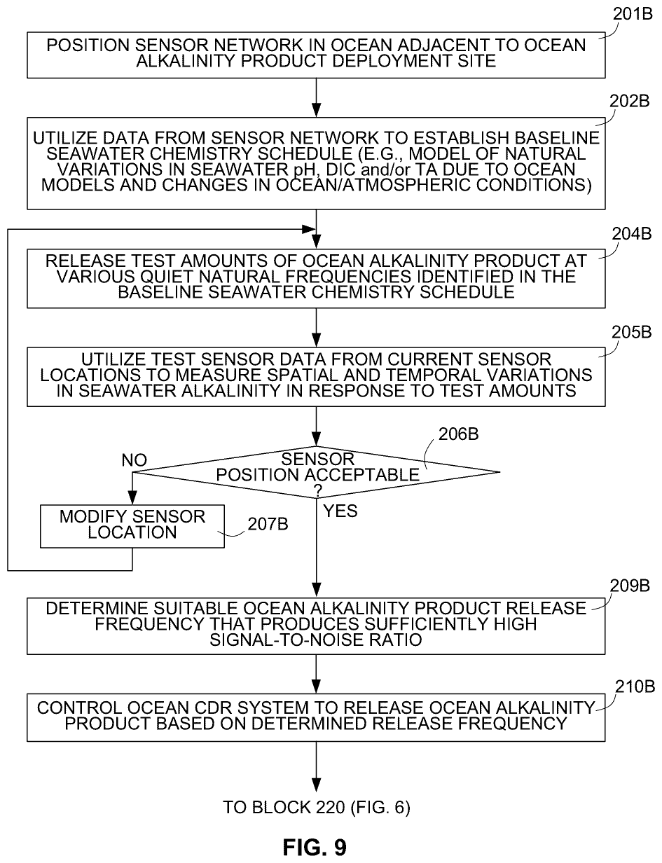

BPED 110 generally includes an electrodialysis (ED) apparatus 130 and a post-production subsystem (PP SUBSYS) 170 whose operations are controlled by control circuit 180. As described in additional detail below, ED apparatus 130 is configured to electrochemically process salt such as sodium chloride (NaCl) provided in feedstock solution 111-IN to generate a base substance such as sodium hydroxide (NaOH) and an acid substance such as hydrochloric acid (HCl), and post-production subsystem 170 is configured to generate ocean alkalinity product 113-OUT containing the base substance generated by ED apparatus 130. BPED 110 also includes flow control resources (described below with reference to FIG. 8) that are configured to direct salt, acid and base solution streams through ED apparatus 130. In the exemplary embodiments described herein, BPED 110 is controlled such that ocean alkalinity product 113-OUT (and, hence, base substance) is released into ocean 50 with a time waveform whose power spectrum contains significant contributions in at least one non-natural (quiet natural variation) frequency (also referred to herein as a quiet natural seawater carbonate chemistry variation frequency). In some embodiments, ocean CDR systems may utilize other base producing devices (i.e., different from ED apparatus 130 and post-production subsystem 170) to generate/release base substance while remaining within the spirit and scope of the invention, provided a base substance generated by the ocean CDR system is released with a time waveform whose power spectrum contains significant contributions in at least one non-natural frequency. BPED 110 is of a size that is capable of generating ocean alkalinity product 113-OUT in a sufficient quantity to increase the alkalinity of a designated ocean area. When OAE system 100 includes two or more BPEDs, each BPED is configured and operates as described below with reference to BPED 110. In some embodiments, feedstock solution 111-IN includes seawater 51 that is pumped directly from ocean 50 through an inflow transfer pipe 53. In other embodiments (not shown), feedstock solution 111-IN may comprise brine generated by a brine source (e.g., a desalination plant or a water recycling plant that processes seawater and generates brine as a byproduct).

ED apparatus 130 generally includes an ion exchange (IE) stack 135 and opposing electrodes (e.g., an anode 138-1 and a cathode 138-2) positioned on opposite sides of IE stack 135. IE stack 135 includes a salt flow channel 131, an acid flow channel 132 and a base flow channel 133, a first ion exchange membrane 134-1 disposed between salt flow channel 131 and acid flow channel 132, and a second ion exchange membrane 134-2 disposed between salt flow channel 131 and base flow channel 133. Salt flow channel 131, an acid flow channel 132 and a base flow channel 133 and are configured to conduct the salt, acid and base solution streams, which are indicated in FIG. 1 using base reference numbers 111, 112 and 113, respectively. Because salt is converted into acid substance and base substance during the electrochemical process, the amount (concentration) of salt in salt solution stream 111 is higher before entering IE stack 135 than after leaving IE stack 135, and the amounts of acid substance in acid solution stream 112 and base substance in base solution stream is lower before entering IE stack 135 and higher upon leaving IE stack 135. For descriptive purposes, inflowing/upstream solution stream portions (i.e., stream portions flowing into the upper/input end of IE stack 135) are indicated with the suffix “−1”, outflowing/downstream solution stream portions are indicated with the suffix “−2”, and the terms “weak” and “strong” are used herein to indicate the relative concentrations of salt, acid and base substances in the upstream/downstream salt, acid and base solution stream portions. That is, to indicate the removal of salt molecules by the electrochemical process described below, upstream salt solution stream portion is referred to as strong salt stream 111-1 and the downstream portion is referred to as weak salt stream 111-2 (i.e., because of the removal of salt molecules by the electrochemical processing described above). In contrast, to indicate the addition of acid/base substance to the acid and base streams by the electrochemical process, the upstream acid/base solution stream portions are referred to as weak acid stream 112-1 and weak base stream 113-1, and the downstream portions are referred to as strong acid stream 112-2 and strong base stream 113-2.

Referring to IE stack 135, the electrochemical process performed by ED apparatus 130 generally involves ionizing salt molecules (NaCl) disposed in strong (inflowing) salt solution stream 111-1 to produce both acid substance (HCl) in strong (outflowing) acid solution stream 112-2 and a base substance (NaOH) in strong (outflowing) base solution stream 113-2. In one embodiment, electrodialysis apparatus 130 includes manifold or other structures (not shown) that are configured to direct strong salt stream 111-1 into the inlet of salt flow channel 131, to direct weak acid stream 112-1 into the inlet of acid flow channel 132, and to direct weak base stream 113-1 into the inlet of base flow channel 133. While solution streams 111-1, 112-1 and 113-1 are passed through flow channels 131, 132 and 133, respectively, a stack voltage V_{STACK} is applied across electrodes 138-1 and 138-2 at a voltage level that produces a stack current I_{STACK}, which is conducted by the flowing solutions and through the intervening ion exchange membranes 134-1 and 134-2 (i.e., as depicted by the dot-line arrow extending from anode 138-1 to cathode 138-2). Referring to salt flow channel 131, when a suitably strong stack current I_{STACK} is conducted through stack 135, at least some of the salt molecules (NaCl) in the portion of salt solution stream 111-1 disposed in flow channel 131 are electrochemically processed (ionized), thereby producing sodium ions Na⁻ and chlorine ions Cl⁺ that are attracted to the positive (V_{STACK}) and negative (ground) potentials applied to electrodes 138-1 and 138-2, respectively. As depicted in acid flow channel 132, ion exchange membrane 134-1 is configured such that chlorine ions Cl⁺ migrate from flow channel 131 through ion exchange membrane 134-1 toward anode 138-1 and combine with hydrogen ions H⁻ to form hydrochloric acid molecules (HCl) in flow channel 132, whereby strong acid stream 112-2 has a higher acid concentration than weak acid stream 112-1. Similarly, as depicted in base flow channel 133, sodium ions Na⁻ migrate from salt flow channel 131 through ion exchange membrane 134-2 toward cathode 138-2 and combine with hydroxide ions OH⁺ to form sodium hydroxide molecules (NaOH) in flow channel 133, whereby strong base stream 113-2 has a higher base concentration than weak base stream 113-1.

Post-production subsystem 170 generally includes at least one ocean alkalinity production (OAP) device 172, zero or more acid production devices (not shown), and at least one flow control device 175. OAP device 172 receives and processes at least a portion of strong base stream 113-2 generated by IE stack 135 and is configured to produce ocean alkalinity product 113-OUT with a predetermined suitable amount of the base substance generated in IE stack 135 (e.g., such that a pH of ocean alkalinity product 113-OUT is higher than that of seawater 51, but at a level that is safe for sea life). In an embodiment OAP device 172 is configured to test and, if needed, mix strong base stream 113-2 with an appropriate quantity of weak salt stream 111-2 (and/or other diluting liquids) such that the resulting ocean alkalinity product 113-OUT is optimized for release into ocean 50. An optional acid production device (not shown) is configured to generate an acid product 112-OUT using at least some of the acid substance provided in strong acid stream 112-2. Flow control device 175 functions to control the volumetric release rate of ocean alkalinity product (base substance) 113-OUT into ocean 50. In an embodiment, flow control device 175 is an electrically operated valve (e.g., a solenoid valve) having an input port configured to receive ocean alkalinity product 113-OUT from OAP device 172, an output port connected to outflow transfer pipe 52, and a control terminal that is coupled to receive a control signal 183 from control circuit 180. When flow control device 175 is in an opened control state, a flow passage is opened between the input and output ports such that ocean alkalinity product 113-OUT flows from OAP 172 to outfall location 50-0 by way of transfer pipe 52. When flow control device 175 is in a closed control state, the flow passage between the input and output ports is blocked (i.e., the flow of ocean alkalinity product 113-OUT to outfall location 50-0 is prevented). As set forth in additional detail below, in one embodiment flow control device 175 is repeatedly cycled between the opened and closed control states (e.g., by way of asserting/de-asserting control signal 183) such that ocean alkalinity product 113-OUT (base substance) is supplied into ocean 50 as a series of released batches, where each released batch includes a quantity of ocean alkalinity product 113-OUT released from outflow transfer pipe 52 during a release portion of each release cycle (i.e., a period of time during which flow control device 175 is maintained in the opened control state and an associated unit quantity of ocean alkalinity product 113-OUT is continuously released into ocean 50), and each pair of sequential released batches is separated in time by an idle portion of each release cycle (i.e., a period of time during which flow control device 175 is maintained in the closed control state such that no ocean alkalinity product 113-OUT is released into ocean 50).

Referring to the upper portion of FIG. 1, control circuit 180 is configured to generate and transmit various control signals that control the operations performed by BPED 110 and other subsystems/devices (not shown) such that OAE system 100 operates in the manner described herein. In the depicted embodiment, control circuit 180 controls the operations of BPED 110 to generate base substance by way of control signals 181, controls the operations of post-production subsystem 170 to generate ocean alkalinity product 113-OUT by way of control signals 182, and controls the opened/closed operating state of flow control device 175 by way of control signal 183. In an exemplary embodiment, control circuit 180 causes flow control device 175 to enter the opened control state by way of asserting control signal 183 (e.g., transmitting a 5V signal to the control terminal of flow control device 175), and control circuit 180 causes flow control device 175 to enter the closed control state by way of de-asserting control signal 183 (e.g., maintaining the control terminal of flow control device 175 at 0V). In one embodiment control circuit 180 is implemented by at least one electronic device (e.g., a computer/processor and/or an application specific integrated circuit device) that implements software-based instructions or is otherwise configured to execute various system-related control algorithms in a fully autonomously manner (i.e., without requiring human operator input). In some embodiments, control circuit 180 is also configured to control BPED 110 during maintenance and other operations in the manner described in co-owned U.S. Pat. No. 11,629,067, entitled “OCEAN ALKALINITY SYSTEM AND METHOD FOR CAPTURING ATMOSPHERIC CARBON DIOXIDE”, which is incorporated herein by reference in its entirety.

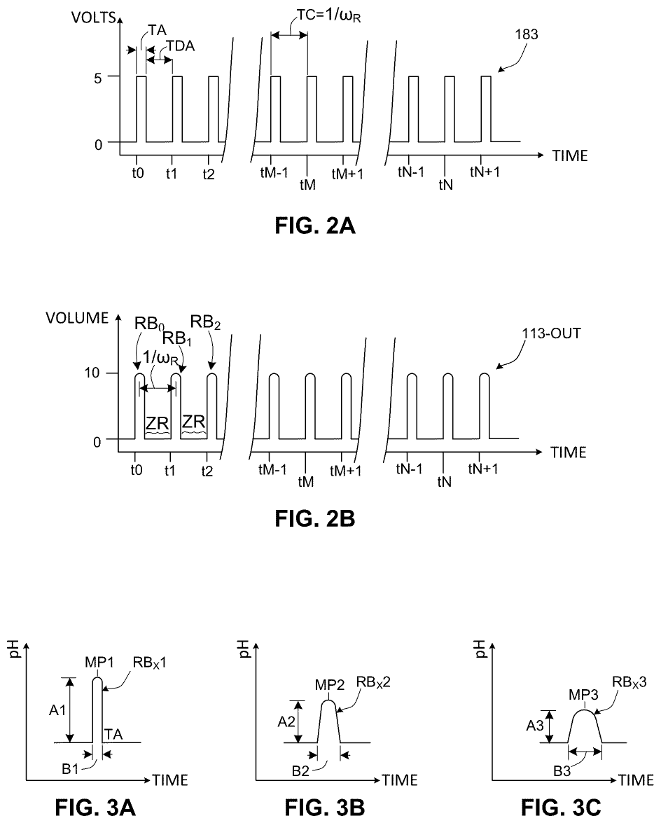

In one embodiment controller 180 is programmed or otherwise configured to control electrochemical OAE system 100 such that ocean alkalinity product 135-OUT (and, hence, the base substance generated by ED apparatus 130) is supplied into ocean 50 as a series of discrete released batches that are sequentially released in accordance with at least one release frequency ω_{R}. In an exemplary embodiment depicted in FIG. 2A, the volumetric release rate of ocean alkalinity product 135-OUT into ocean 50 is controlled by way of cycling (i.e., alternately asserting and de-asserting) control signal 183 such that the operating state of flow control device 175 alternates between opened and closed in accordance with selected release frequency ω_{R} (i.e., with a cycle time period TC that is equal to 1/ω_{R}), whereby a relatively large (non-zero) volume of ocean alkalinity product 135-OUT is supplied to ocean 50 during the asserted (release) portion TA of each release cycle, and a relatively small volume (zero or insignificant volume) of ocean alkalinity product 135-OUT is supplied to ocean 50 during the de-asserted (idle) portion TDA of each release cycle. Referring to FIG. 2B, by cycling the opened/closed operating state of flow control device 175 using the depicted timing of control signal 183 (shown in FIG. 2A), ocean alkalinity product 113-OUT is supplied into ocean 50 in a series of discrete released batches RB₀, RB₁ . . . RB_{N+1}, where successive released batches (e.g., released batched RB₀ and RB₁) are spaced in time by cycle time period 1/ω_{R}. Although depicted using a square wave in FIG. 2A, release frequency ω_{R} can be generated according to any periodic waveform (e.g., sinusoidal, triangular, saw tooth or pulse), and that the asserted period TA of each cycle may be equal to, longer or shorter that the de-asserted period TDA of each cycle. In some embodiments, each released batch comprises a substantially equal (unit) volumetric amount of ocean alkalinity product 113-OUT (e.g., 100 gallons per release cycle), and each released batch is preceded and succeeded by a zero (or insignificant) release period (e.g., as indicated in FIG. 2B, released batch RB₁ is preceded and succeeded by zero release periods ZR).

Referring again to FIG. 1, each released batch RB₀, RB₁ . . . RB_{N+1} comprises a volumetric amount of ocean alkalinity product (base substance) 113-OUT that is passed through flow control device 175 during an associated opened control state and is supplied from transfer pipe 52 into ocean 50 at outfall location 50-0. Because each released batch is discrete, the radial diffusion of base material contained within each released batch generates a region of increased alkalinity that gradually propagates in a wave-like manner away from outfall location 50-0 (e.g., along dispersion path DP). For illustrative purposes, the dispersion of base substance associated with a series of discrete released batches RB₀ to RB_{N+1} along a dispersion path DP is depicted in FIG. 1 at time tN+1 by a corresponding shaded (i.e., relatively dark) region in seawater 51. The series of discrete released batches RB₀ to RB_{N+1} includes an initial released batch RB₀ released at an initial time t0 that reaches location 50-3 at time tN+1, a second released batch RB₁ released at time t1 (i.e., a fixed period of time after time t0, where the fixed period is determined by release frequency ω_{R}) that has not yet reached location 50-3 at time tN+1, a subsequently released batch series RB_{M-1} to RB_{M+1} including released batch RB M that reaches location 50-2 at time tN+1, and so on until a last (most-recently) released batch RB_{N+1} is released at outfall location 50-0 at time tN+1.

As indicated by dashed-line arrows P extending from last released batch RB_{N+1}, the base material contained within each released batch radially propagates (diffuses in seawater 51) away from outfall location 50-0 upon entering ocean 50. The dispersion of the base substance associated with penultimate released batch RB_{N} is depicted as an associated semi-circular shell-like dark region that is generally concentrically disposed around last released batch RB_{N+1} and is separated from last released batch RB_{N+1} by a gap region G, where gap region G represents a region of lower base substance concentration that occurs between the wave-like higher base substance concentration associated with penultimate released batch RB_{N} and the higher base concentration associated with last released batch RB_{N+1}. The lower base substance concentration in gap region G is generated by the closed operating state of flow control device 175 occurring between the two opened operating states associated with released batches RB_{N} and RB_{N+1} (i.e., the idle release cycle portion occurring between times t_{N} and t_{N+1} during which control signal 183 is de-asserted). Similarly, the dispersive progress of discrete released batches RB₀ to RB_{N-1} along dispersion path DP is indicated by corresponding dark curved regions separated by intervening gaps, representing wavefront sections of the wave-like base substance dispersion associated with each released batch.

For clarity and brevity, it is assumed that the base substance of each released batch RB₀ to RB_{N+1} propagates away from outfall location 50-0 at a substantially uniform average rate. That is, after each discrete released batch of ocean alkalinity product 113-OUT exits the outlet of transfer pipe 52 and enters ocean 50, the base substance contained within each released batch undergoes molecular diffusion into seawater 51, thereby causing a corresponding change in seawater chemistry (e.g., alkalinity). The base substance contained in the series of released batches RB₀ to RB_{N+1} then propagates (disperses) in the surrounding seawater 51 away from outfall location 50-0 (e.g., along dispersion path DP) in a wave-like manner, whereby the base substance of each released batch forms an associated discrete wavefront. That is, as each released batch RB₀ to RB_{N-1} propagates through seawater 51, its associated wavefront (wave-like region of higher base substance concentration) produces a corresponding increase-then-decrease in the time-based seawater chemistry measurement data at each sensor deployment location along dispersion path DP (i.e., as the base substance wavefronts sequentially pass the given point). When seawater carbonate chemistry data is collected and processed as set forth below, a time waveform of the collected seawater chemistry exhibits a power spectrum containing significant contributions that correspond with each base substance wavefront. Because each released batch RB₀ to RB_{N+1} propagates away from outfall location 50-0 at a substantially uniform rate, the time waveform of the collected seawater chemistry would indicate the time delay between each sequential pair of detected wavefronts. For example, if the base substance wavefront of released batch RB_{N-2} is detected at location 50-1 one-half hour before the base substance wavefront of released batch RB_{N-1} is detected at location 50-1, then flow control 175 was controlled to generate released batch RB_{N-2} one-half hour before generating released batch RB_{N-1}.

The base substance wavefront of released batches RB₀ to RB_{N-1} undergoes weakening and distortion due to dispersion and dilution of the base substance as released batches RB₀ to RB_{N-1} propagate away from outfall location 50-0. This weakening and distortion is depicted in FIGS. 3A to 3C, which show exemplary seawater pH time waveforms indicating measurements taken at three distances from outfall location 50-0 in response to (only) the base substance wavefront of exemplary released batch RB_{X} (i.e., all changes to seawater pH caused by natural variations are ignored for descriptive purposes). That is, FIG. 3A depicts a seawater pH time waveform RB_{X}1 produced by the base substance wavefront of exemplary released batch RB_{X} as it passes a sensor deployment location 50-1 (see FIG. 1) that is a relatively short distance D1 from outfall location 50-0, FIG. 3B depicts a time waveform RB_{X}2 produced as the base substance wavefront passes a location 50-2 (see FIG. 1) that is an intermediate distance D2 from outfall location 50-0, and FIG. 3C depicts a time waveform RB_{X}3 produced as the base substance wavefront passes a location 50-3 that is a relatively long distance D3 from outfall location 50-0. Note that the shape of time waveforms RB_{X}1 to RB_{X}3 is greatly exaggerated and distorted for descriptive purposes (e.g., the trailing edge decreases at a substantially flatter angle than the leading edge in actual time waveforms). As indicated in FIG. 3A, time waveform RB_{X}1 has a relatively small bandwidth B1 and a relatively large peak amplitude (pH value) A1, which indicates that the base substance waveform associated with released batch RB_{X} remains relatively compact (i.e., the base substance has not dispersed/diluted significantly after propagating relatively short distance D1 from outfall location 50-0). FIG. 3B indicates that further propagation of released batch RB_{X} to intermediate distance D2 from outfall location 50-0 subjects the base substance wavefront to significantly more dispersion/dilution, whereby time waveform RB_{X}2 has a somewhat broader bandwidth B2 and a relatively smaller peak amplitude A2. FIG. 3C depicts a time waveform RB_{X}3 for released batch RB_{X} after its base substance wavefront has further propagated to distance D3, producing an even broader bandwidth B3 and an even smaller peak amplitude A3 due to the further dispersion of base substance. The gradual weakening and distortion of the base substance wavefronts is also depicted in FIG. 1 by the relatively thick dark curved regions near outfall location 50-0 and relatively thin dark curved regions far from outfall location 50-0. For example, released batch RB_{N-1} is indicated by a relatively thick dark curved region, indicating its base substance wavefront remains relatively coherent and dense (narrow and tall, as depicted in FIG. 3A) at relatively short distance D1. Conversely, released batch RB₀ is indicated by a relatively thin dark curved region, indicating its base substance wavefront has weakened and distorted (e.g., as depicted in FIG. 3C). The gradual weakening and distortion of each released batch continues as its base substance wavefront propagates further from outfall location 50-0, and at some distance (i.e., depending on the base substance amount associated with each released batch) the peak amplitude of base substance becomes too distorted/diluted to detect and/or distinguish from natural seawater chemistry variations. However, as described further below, within the effective range of OAE system 100 (i.e., within a region around outfall location 50-0 within which the peak amplitude can be distinguished from natural seawater chemistry variations), the detection of periodic increases in seawater pH level that coincide with release frequency ω_{R} (i.e., corresponding to the detection of base substance wavefronts associated with released batches RB₀ to RB_{N-1}) can be used to verify the contribution of electrochemical OAE system 100 to atmospheric CO₂ reduction/removal.

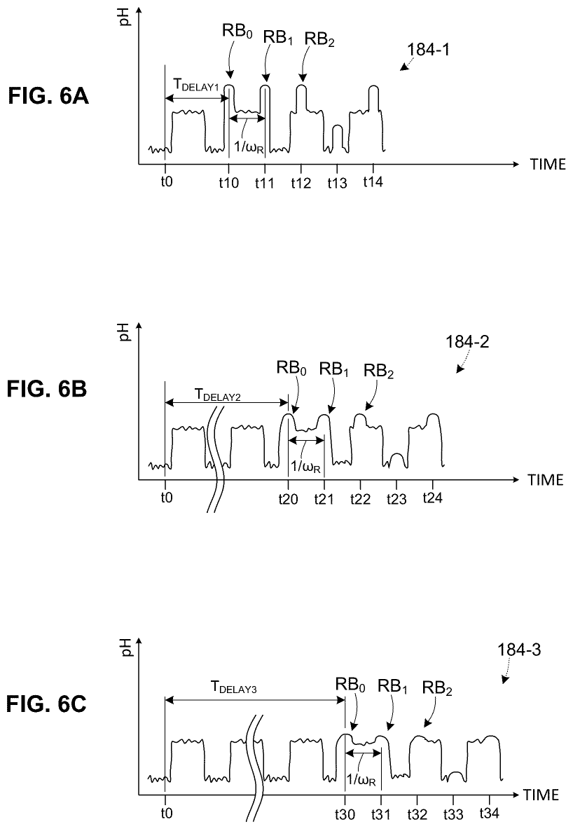

In the exemplary embodiment depicted in FIG. 1, ocean-based sensors S1, S2 and S3 collectively form a sensor network 185 that is utilized to detect the base substance wavefronts generated by electrochemical OAE system 100. To facilitate this detection, each sensor S1, S2 and S3 is configured to measure one or more seawater carbonate chemistry parameters that are change in accordance with the concentration of the base material associated with each base substance wavefront (e.g., one or more of pH, dissolved organic carbon (DIC), partial pressure of CO₂ (PCO₂) and total alkalinity (TA) of seawater 51), and is configured to generate and transmit corresponding time-based ocean chemistry data in a form that can be processed by control circuit (processing device) 180 in the manner described below. In the exemplary embodiment, ocean-based sensor S1, S2 and S3 are tethered or otherwise maintained in dispersion path DP at corresponding distances D1, D2 and D3 from outfall location 50-0 (i.e., at sensor deployment locations 50-1, 50-2 and 50-3, respectively) such that each sensor S1, S2 and S3 measures and transmits time-based seawater chemistry data collected at corresponding different distances from outfall location 50-0. For example, sensor S1 may be configured to measure seawater pH at ocean region (sensor deployment location) 50-1 located a relatively short distance D1 from outfall location 50-0 and to generate time-based pH measurement data 184-1 in accordance with these measurements, sensor S2 may be configured to generate time-based pH measurement data 184-2 from a second ocean region 50-0 located an intermediate distance D2 from outfall location 50-0, and sensor S3 may be configured to generate time-based pH measurement data 184-3 from a third ocean region 50-3 located a relatively long distance D3 from outfall location 50-0. Simplified examples of time-based pH measurement data 184-1, 184-2 and 184-3 are described below with reference to FIGS. 6A to 6C. In the exemplary embodiment, seawater chemistry data 184-1, 184-2 and 184-3 is transmitted from sensors S1, S2 and S3 (e.g., using wireless or wired transmission) directly to control circuit 180, whereby sensors S1, S2 and S3 may be considered a part of OAE system 100. In other embodiments, the seawater chemistry data generated by sensors S1, S2 and S3 may be transmitted and processed by another processing device (not shown), whereby sensors S1, S2 and S3 may be considered separate from (not a part of) OAE system 100. Although three sensors are utilized in the exemplary embodiment, in other embodiments (not shown) any number of sensors (e.g., one, two, four, . . . ) may be utilized to collect time-based ocean chemistry data.

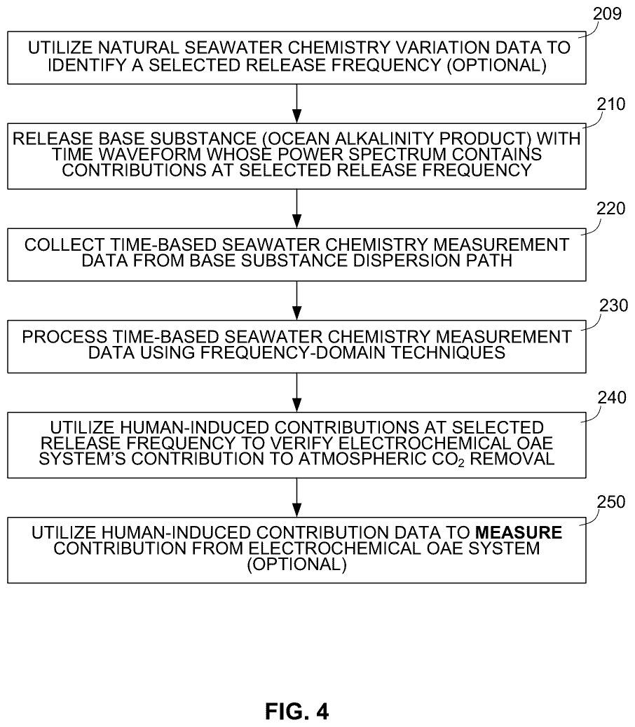

FIG. 4 is a flow diagram depicting a frequency-based base substance detection method for reliably verifying (and optionally measuring) the contribution of electrochemical OAE system 100 (shown in FIG. 1) to atmospheric CO₂ removal. In one embodiment, the method includes controlling electrochemical OAE system 100 to release base substance (ocean alkalinity product) 113-OUT into ocean 50 as a series of discrete released batches RB₀ to RB_{N+1} in accordance with a selected non-natural release frequency ω_{R} (block 210), collecting time-based seawater chemistry measurement data from one or more of ocean-based sensors S1, S2 and S2 (block 220), processing the seawater chemistry measurement data using frequency-domain techniques to generate frequency-based data (block 230), and then utilizing the frequency-based data to verify human-induced changes in the seawater chemistry occurring at the selected non-natural release frequency ω_{R} (block 240). In some embodiments the method also includes a preliminary process of utilizing natural seawater chemistry variation data to identify a suitable selected release frequency ω_{R} (block 209). In some embodiments the method includes a post-verification process of using the frequency-based data indicating human-induced seawater chemistry changes to measure the contribution of electrochemical OAE system 100 to atmospheric CO₂ removal (block 250). Each process of the method is described in additional detail in the following paragraphs.

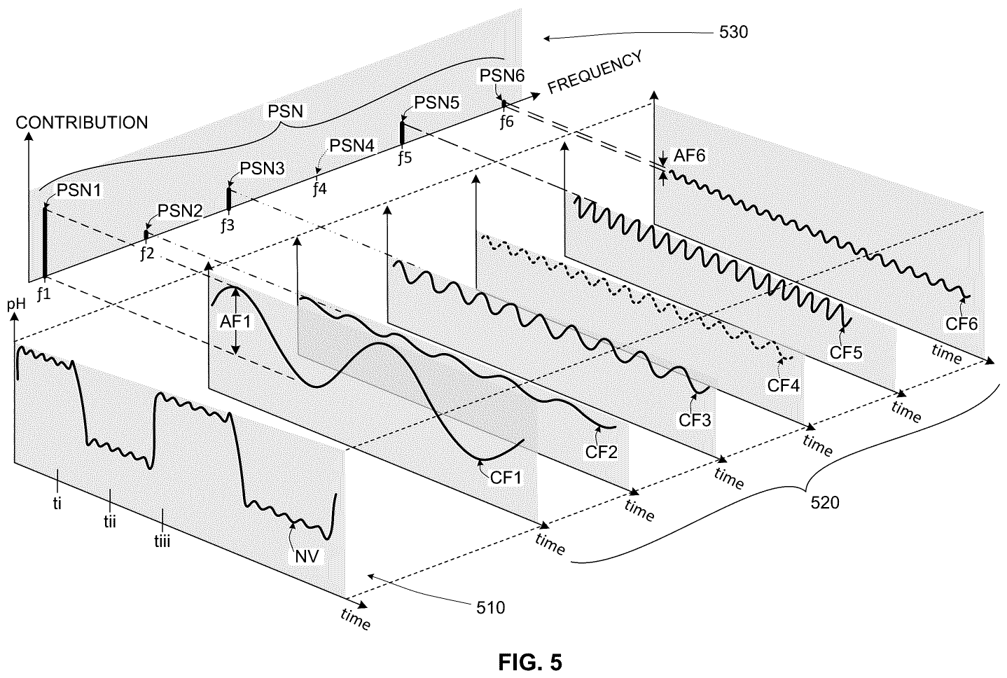

Referring to block 209 at the top of FIG. 4, in some embodiments the natural seawater chemistry variation data (also referred to herein as a baseline seawater chemistry schedule) is used to identify one or more selected release frequencies before the selected release frequency(ies) is/are utilized to control the timing between release batches (i.e., the release of base substance per block 210). Referring to FIG. 1, ocean-based sensors S1 to S3 may be utilized to collect preliminary time-based seawater carbonate chemistry measurement data from ocean 50 at corresponding locations 50-1 to 50-3 along dispersion path DP before operation of OAE system 100 is initiated (e.g., before any base substance generated by BPED 110 has been supplied to seawater 51). As explained below with reference to graph 510 (FIG. 5), all changes in the seawater carbonate chemistry parameter levels that appear in the preliminary time-based seawater carbonate chemistry measurement data are assumed to be caused solely by natural seawater chemistry variations. In a manner similar to that described above with reference to block 230 (FIG. 4) and described below with reference to graphs 520 and 530 (FIG. 5), the preliminary seawater carbonate chemistry measurement data is then processed using frequency-domain techniques to generate a natural seawater carbonate chemistry variation power spectrum. As described below with reference to graph 530 (FIG. 5), one or more quiet frequencies (i.e., one or more frequencies exhibiting insignificant power spectra levels) is/are identified in the natural seawater carbonate chemistry variation power spectrum, and then the selected one or more quiet frequencies is/are utilized as the selected release frequency(ies) during subsequent ocean CDR system operations.

FIG. 5 is multi-part diagram depicting in a greatly simplified manner how frequency-domain techniques (e.g., Fourier transform) may be utilized to identify/select release frequencies using natural seawater chemistry variation data according to an embodiment of the present invention. The multi-part diagram includes a graph 510 indicating exemplary time-based natural seawater pH variation waveform NV, graphs 520 depicting associated component frequency waveforms CF1 to CF6 that collectively describe natural seawater pH variation waveform NV, and graph 530 depicts a natural pH variation power spectrum PS generated using component frequency waveforms CF1 to CF6. Note that the shape of time-based natural seawater pH variation waveform NV (graph 510) is selected solely for descriptive purposes and is not intended to represent actual natural seawater pH variation measurements. Similarly, note the number of component frequencies depicted by frequency waveforms CF1 to CF6 (graphs 520) and natural pH variation power spectrum PSN (graph 520) are limited to six for brevity and clarity, and that the frequency-domain representation of actual natural seawater pH variations is likely to include contributions at a significantly larger number of component frequencies.

Referring to graph 510 (lower left side of FIG. 5), natural seawater pH variation waveform NV indicates exemplary time-based variations in seawater pH levels caused by naturally occurring changes in ocean/atmospheric conditions, and represents data collected by ocean-based sensor configured to measure seawater pH levels. It is established that seawater pH (and other seawater carbonate chemistry parameters) undergo natural time-based variations that are caused by a wide range of natural processes (see, for example, Earth Syst. Sci. Data, 11, 421-439, 2019, G. E. Hofmann et al., High-Frequency Dynamics of Ocean pH: A Multi-Ecosystem Comparison; PLoS ONE 6:11 (2011), Scientific Reports volume 11, Article number: 505/2 (2021)). Natural seawater pH variation waveform NV depicts an exemplary sequence of time-based seawater pH levels that reflect the continuous influence (i.e., increase and decrease) of natural changes in ocean/atmospheric conditions, thereby producing relatively high seawater pH levels during some time periods (e.g., at times ti and tiii) and relatively low seawater pH levels during other time periods (e.g., at time tii). In alternative embodiments, natural seawater pH variation waveform NV may represent preliminary time-based seawater carbonate chemistry measurement data collected, for example, by one or more of sensors S1 to S3 (FIG. 1) or may represent preliminary time-based seawater carbonate chemistry measurement data procured from another source.

Graphs 520 and 530 depict the conversion of natural seawater pH variation waveform NV (graph 510) into corresponding natural pH variation power spectrum PS using one or more frequency-domain techniques (e.g., Fourier transform conversion). Power spectrum PS (graph 530) is a frequency-based representation including power spectra values PSN1 to PSN6 that indicate the relative contributions at frequencies f1 to f6 to natural seawater pH variation waveform NV. Component frequency waveforms CF1 to CF6 (graphs 520) represent an intermediate phase in the conversion process showing that the relative contribution at each frequency f1 to f6, which correspond to power spectra values PSN1 to PSN6, may be represented by time-based waveforms whose amplitudes correspond to associated power spectra values. For example, the relatively large contribution at relatively low frequency f1 (corresponding to power spectra value PSN1) is depicted by the relatively large amplitude AF1 of component frequency waveform CF1. Conversely, the relatively small contribution at relatively high frequency f6 (corresponding to power spectra value PSN6) is depicted by the relatively small amplitude AF6 of component frequency waveform CF6. In a similar fashion, the relative contributions at intermediate frequencies f2, f3 and f5 (corresponding to power spectra values PSN2, PSN3 and PSN5, respectively) are depicted by the corresponding amplitudes of component frequency waveforms CF2, CF3 and CF5. That is, when the natural component frequencies indicated by frequency waveforms CF1 to CF6 are combined, the resulting waveform would correspond with natural seawater pH variation waveform NV (graph 510). Note that the dotted line indicating component frequency waveform CF4 indicates that, for explanatory purposes, the contribution at frequency f4 (corresponding to power spectra value PSN4) is assumed to be insignificant (e.g., zero).

As mentioned above, the release frequency(s) utilized to control the batch release of base substance from an ocean CDR system preferably coincides with relatively quiet frequency(s) in the natural pH variation power spectrum signal. Referring to exemplary natural pH variation power spectrum PSN (graph 530, FIG. 5), a suitable quiet natural frequency occurs at frequency f4 because its associated spectra power value PSN4 is insignificant (e.g., zero). That is, spectra power peak PSN4 indicates that substantially zero power exists for natural variation at frequency f4, thereby maximizing signal-to-noise ratio when the ocean CDR system utilizes frequency f4 as the selected release frequency. In some embodiments the selected release frequency may correspond with one or more relatively quiet natural variation frequencies (e.g., component frequencies f2 or f6, indicated by relatively short spectra power peaks PSN2 and PSN6, respectively).

After natural seawater chemistry variation data is utilized to identify and select a non-natural (quiet natural variation) frequency for use as the selected release frequency (FIG. 4, block 209), subsequent control of the ocean CDR system using the selected release frequency (FIG. 4, block 210) causes the release of seawater alkalinity product with a time waveform whose power spectrum contains contributions at quiet natural seawater carbonate chemistry variation frequency. As depicted in FIG. 1, when control circuit 180 generates control signal 183 using a selected release frequency ω_{R}, which coincides with non-natural frequency f4, flow control device 175 releases ocean alkalinity product 113-OUT in a way that generates discrete base substance wavefronts (released batches) RB₀ to RB_{N+1}. As base substance concentration wavefronts RB₀ to RB_{N+1} propagate past sensors S1 to S3, each sensor generates time-based seawater chemistry data that registers changes in one or more seawater carbonate chemistry parameter levels (e.g., pH, DIC, pCO₂ and/or TA of seawater 51 at each sensors location.

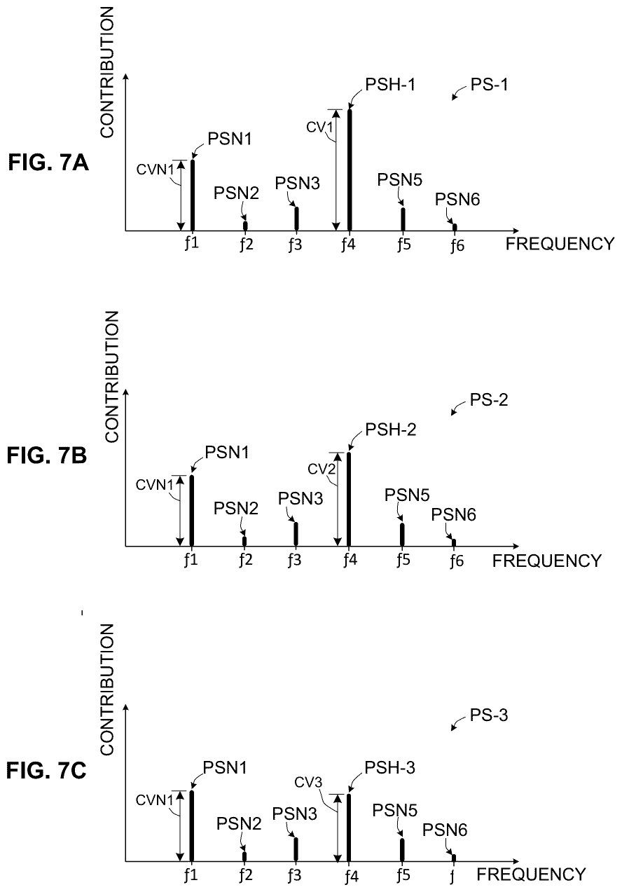

FIGS. 6A, 6B and 6C respectively depict simplified examples of partial time-based pH measurement data 184-1, 184-2 and 184-3 collected by sensors S1, S2 and S3, respectively, during the base release operations by electrochemical OAE system 100 according to the example described above with reference to FIG. 1. Note that, for purposes of describing the time-based seawater carbonate chemistry changes measured by sensors S1, S2 and S3, the depictions of time-based pH measurement data 184-1, 184-2 and 184-3 utilize (i) the greatly simplified natural seawater variation data described above with reference to FIG. 5 and (ii) the greatly simplified/distorted seawater pH time waveforms described above with reference to FIGS. 3A to 3C (i.e., time-based pH measurement data 184-1, 184-2 and 184-3 are not intended to represent actual sensor data). As mentioned above, initial base substance wavefront RB₀ is generated at time t0 and propagates away from outfall location 50-0 at a substantially constant average rate. Because sensors S1, S2 and S3 are respectively located at progressively longer distances D1, D2 and D3 from outfall location 50-0, initial base substance wavefront RB₀ arrives at sensor S1 (location 50-1) after a first (relatively short) time delay, then arrives at sensor S2 (location 50-2) after a second (longer) time delay, and finally arrives at sensor S3 (location 50-3) after a third (relatively long) time delay. These time delays indicated in FIGS. 6A to 6C as time delay T_{DELAY1} in time-based pH measurement data 184-1 (FIG. 6A), time delay T_{DELAY2} in time-based pH measurement data 184-2 (FIG. 6B), and time delay T_{DELAY3} in time-based pH measurement data 184-3 (FIG. 6C). As indicated by the shape of time-based pH measurement data 184-1, 184-2 and 184-3 the seawater pH data collected by sensors S1, S2 and S3 is influenced solely by natural seawater variations during these time delays (i.e., before the arrival of initial base substance wavefront RB₀). In one embodiment, the data collected during these time delay periods (e.g., between time t0 and time t10 in FIG. 6A) is omitted from subsequent frequency-domain conversion. Referring again to FIG. 1, base substance wavefronts RB₀ to RB_{N+1} are generated in accordance with selected release frequency ω_{R}, and therefore propagate along dispersion path DP and arrive at each of sensors S1, S2 and S3 with a frequency that corresponds to selected release frequency ω_{R}. Referring to time-based pH measurement data 184-1 (FIG. 6A), beginning at time t10 the seawater pH data collected by sensor S1 is influenced both by natural seawater variations and by the human-induced contributions provided by the series of base substance wavefronts. That is, time-based pH measurement data 184-1 reflects the detection of initial base substance wavefront RB₀ at time t10, the detection of a second base substance wavefront RB₁ at time t11, the detection of a third base substance wavefront RB₂ at time t12, and so on. Note that, for descriptive purposes, the time period between the arrival of each sequential pair of base substance wavefronts is assumed to be determined by selected release frequency ω_{R} (e.g., the period between times t10 and t11 is 1/ω_{R}). Similarly, time-based pH measurement data 184-2 (FIG. 6B) reflects both natural and human-induced contributions beginning at time t20, and time-based pH measurement data 184-3 (FIG. 6C) reflects both natural and human-induced contributions beginning at time t30, where the time period between the arrival of each sequential pair of base substance wavefronts is also 1/ω_{R}. As described above with reference to FIGS. 3A to 3C, the base substance concentration of initial base substance wavefront RB₀ gradually decreases as it propagates further away from outfall location 50-0, whereby the human-induced change in seawater pH caused by initial base substance wavefront RB₀ at location 50-1 is greater than the corresponding changes at locations 50-2 and 50-3. This gradual reduction in base substance concentration level is depicted in FIGS. 6A to 6C by the incremental distortion of time-based pH measurement data 184-1, 184-2 and 184-3 at points associated with the arrival of the base substance wavefronts. For example, referring to FIG. 6A, the shape of time-based pH measurement data 184-1 at times t10, t11 and t12 suggests that base substance wavefronts RB₀, RB₁ and RB₂ respectively have the relatively high base substance concentration described above with reference to FIG. 3A when they arrive at location 50-1. Similarly, the shape of time-based pH measurement data 184-2 at times t20, t21 and t22 indicates that base substance wavefronts RB₀, RB₁ and RB₂ have the reduced base substance concentrations described above with reference to FIG. 3B, and the shape of time-based pH measurement data 184-3 at times t30, t31 and t32 indicates that base substance wavefronts RB₀, RB₁ and RB₂ have the even lower base substance concentrations described above with reference to FIG. 3C. FIGS. 7A, 7B and 7C respectively depict exemplary power spectrums PS-1, PS-2 and PS-3, which are generated by respectively processing time-based pH measurement data 184-1 (FIG. 6A), 184-2 (FIG. 6B) and 184-3 (FIG. 6C) using frequency-domain techniques. For purposes of describing key aspects of the present invention, power spectrums PS-1, PS-2 and PS-3 are limited to the same range of frequencies (i.e., f1 to f6) that are introduced above with reference to natural seawater pH variation power spectrum PSN (FIG. 5).

As discussed above, when electrochemical OAE system 100 is configured to release base substance at quiet natural variation (selected release) frequency f4 and the resulting human-induced contributions from base substance wavefronts RB₀ to RB_{N+1} are included in the time-based data collected by sensors S1, S2 and S3, and then Fourier transform conversion is performed on the subsequently generated time-based pH measurement data 184-1, 184-2 and 184-3, the human-induced contributions to seawater pH levels will produce corresponding “unnatural” peak (non-zero power spectra) values at selected release frequency f4. For example, power spectrum PS-1 (FIG. 7A) includes human-induced contribution PSH-1 at selected release frequency f4, power spectrum PS-2 (FIG. 7B) includes human-induced contribution PSH-2 at selected release frequency f4, and power spectrum PS-3 (FIG. 7C) includes human-induced contribution PSH-2 at selected release frequency f4. Note that the “natural” peak values at the other frequencies (e.g., peak value PSN1 at frequency f1, peak value PSN2 at frequency f2, peak value PSN3 at frequency f3, peak value PSN5 at frequency f5 and peak value PSN6 at frequency f6) in each of power spectrums PS-1, PS-2 and PS-3 represent contributions from the natural seawater pH variations described above with reference to FIG. 5.

As described above with reference to FIGS. 3A to 3C, the human-induced change in seawater pH caused by base substance wavefronts RB₀ to RB_{N+1} at location 50-1 is greater than the corresponding changes at locations 50-2 and 50-3, whereby a contribution value (strength) CV1 of human-induced contribution PSH-1 (FIG. 7A) may be greater than contribution value CV2 of human-induced contribution PSH-2 (FIG. 7B), which in turn may be greater than contribution value CV3 of human-induced contribution PSH-2 (FIG. 7C). In contrast, little or no change may occur with respect to the contributions from natural seawater pH variations (e.g., natural peak value PSN5 may have the same contribution value CVN1 in each power spectrum PS-1, PS-2 and PS-3.

Note that any of power spectrum PS-1, PS-2 and PS-3 may be used to verify the contributions of and ocean CDR system (e.g., electrochemical OAE system 100, shown in FIG. 1). That is, because OAE system 100 is operated using a non-natural release frequency f4, the signal-to-noise ratio in the seawater pH measurement data is greatly enhanced, even when relatively weak spectra power peaks (e.g., human-induced peak PSH-3, FIG. 7C) are detected, because the selected release frequency f4 is a quiet natural variation frequency (i.e., even human-induced peak PSH-3 provides a clear human-induced variation data “signal” that can be easily distinguished from the natural variation “noise”). Moreover, the strengths (contribution values) CV1, CV2 and CV3 of human-induced contributions PSH-1, PSH-2 and PSH-3 may be used to measure the contribution of electrochemical OAE system 100 to atmospheric CO₂ removal at each distance D1 to D3 from outfall location 50-0.

FIG. 8 shows a partial electrochemical OAE system including a BPED 110A, which represents a more detailed embodiment of BPED 110 (shown in FIG. 1) and is provided for purposes of describing in greater detail the base substance generating operations performed by electrochemical OAE system of the type associated with the disclosed invention (e.g., OAE system 100, shown in FIG. 1). BPED 110A includes a fluid storage system 120A, an electrodialysis apparatus 130A, a post-production subsystem 170A and a flow control system comprising various pumps, valves, and a series of flow lines, which are described below. In one embodiment BPED 110A operates in accordance with control signals generated by and transmitted from an OAE system controller (not shown) to generate and release ocean alkaline product 113A-OUT. In some embodiments, operations of BPED 110A are further controlled such that a base production efficiency of BPED 110A (i.e., an efficiency with which the base substance contained within alkaline product 113A-OUT is produced) is optimized in the manner described in co-owned U.S. patent application Ser. No. 18/131,839, entitled “PRODUCTION EFFICIENCY OPTIMIZATION FOR BIPOLAR ELECTRODIALYSIS DEVICE”, which is incorporated herein by reference in its entirety.

Referring to the upper portion of FIG. 8, fluid storage system 120A can include at least three main holding tanks: a salt (first) holding tank 121A-1 utilized to receive and store (buffer) a quantity of feedstock (salt) solution 111A, an acid (second) holding tank 121A-2 utilized to store a quantity of acid solution 112A, and a base (third) holding tank 121A-3 utilized to store a quantity of base solution 113A. In an embodiment, each holding tank 121A-1 to 121A-3 can be implemented using a standard 1000 L IBC caged tote tank, where each holding tank 121A-1 to 121A-3 includes an associated inflow port and an associated outflow port. In other embodiments (not shown), fluid storage system 120A may be modified to include one or more additional holding tanks that may be utilized to store, for example, fresh or deionized water or intermediate solutions utilized by BPED 110A. In some embodiments additional holding tanks may be utilized to store quantities of previously generated base substance and acid substance solutions, thereby allowing the control algorithm to determine the best time to generate acid and base substances (e.g., when electricity carbon intensity and price are most favorable) from the best time to supply ocean alkalinity product 113A-OUT to ocean 50 (e.g., as determined at least partially by data 184-1 from ocean-based seawater chemistry sensor S1, which is located adjacent to outfall location 50-1).

In some embodiments BPED 110A includes a pretreatment unit 140A connected between the conduit supplying feedstock solution 111A-IN (e.g., seawater 51 from ocean 50 or brine from another feedstock source) and fluid storage system 120A. Pretreatment unit 140A receives and processes feedstock solution 111A-IN and is configured to generate both a reduced-salt fluid 115A (e.g., permeate having essentially 0% salt) and a high-salt fluid 111A-0 (concentrate having approximately 7% salt), whereby high-salt fluid 111A-0 has a higher salt concentration than both seawater 51 and reduced-salt fluid 115A. As discussed below, high-salt fluid 111A-0 is directed to first (salt) holding tank 121A-1 and reduced-salt fluid 115A is directed to second (acid) holding tank 121A-2 and the third (base) holding tank 121A-3 to replace the outflow volumes represented by salt product fraction 111A-22, acid product fraction 112A-22 and base product fraction 113A-22. In an exemplary embodiment pretreatment unit 140A may be implemented by a commercially available reverse osmosis system.

Electrodialysis apparatus 130A includes an AC/DC converter (stack current generator) 139A configured to generate a stack voltage V_{STACK} across electrodes 138A-1 and 138A-2, thereby generating a stack current I_{STACK} passing through an IE stack 135A sandwiched between electrodes 138A-1 and 138A-2. As described above with reference to FIG. 1, IE stack 135A generally includes at least one cell formed by a salt chamber 131A disposed between an acid chamber 132A and a base chamber 133A and separated by ion exchange membranes 134A-1 and 134A-2. IE stack 135A may include multiple cells disposed in a repeating series arrangement, where salt, acid and base solution streams are split into associated sub-streams by an input manifold and passed through the salt, acid and base chamber of each cell, for example, using the arrangement described with reference to FIG. 4 of U.S. Pat. No. 11,629,067 (cited above). During operation, stack current I_{STACK} passes through all of the multiple cells to electrochemically process NaCl (salt) atoms provided in each of the salt sub-streams, thereby enhancing (i.e., decreasing the pH of) an adjacent acid solution sub-stream and enhancing (i.e., increase the pH of) an adjacent base solution sub-stream. In one embodiment, AC/DC converter 139A is configured to convert AC stack power P_{STACK-AC} into direct current (DC) stack power P_{STACK-DC}, and AC/DC converter 139A is configured to control the amount (level) of both DC stack voltage V_{STACK} and DC stack current I_{STACK} supplied to IE stack 135A in response to control signals (not shown) received from control circuit 180. In other embodiments AC/DC converter 139A may be replaced with another circuit capable of generating I_{STACK} at a current level (amount) determined by an applied control signal.

In one embodiment, the flow control system of BPED 110A comprises various control elements (e.g., pumps and valves) that are collectively configured to direct streams of the various solutions (and other fluids) by way of associated conduits (flow lines) in the manner depicted in FIG. 8. For example, a set of input pumps 145A-1 (indicated by circled arrows pointing along the associated flow direction) includes a first feedstock pump 145A-11 that supplies feedstock solution 111A-IN by way of an input conduit to pretreatment unit 140A, a second pump 145A-12 that supplies concentrate 111A-0 by way of a conduit 155A-11 to holding tank 121A-1, and one or more additional pumps that supply permeate 115A to holding tanks 121A-2 and 121A-3 by way of conduits 155A-12 and 155A-13, respectively. A set of isolation valves 146A-1 are operably connected in conduits 155A-21 and 155A-23 between isolation valves 146A-1 and IE stack 130A, and are controlled by way of associated control signals (e.g., generated by controller 180, FIG. 1) to open or close the flow of strong salt stream 111A-1 from holding tank 121A-1 to salt chamber 131A by way of conduit 155A-21, the flow of weak acid solution 112A-1 from holding tank 121A-2 to base chamber 132A by way of conduit 155A-22, and the flow of weak base solution 113A-1 from holding tank 121A-3 to base chamber 133A by way of conduit 155A-23. A set of pressure control pumps 145A-2 are operably connected in conduits 155A-21 and 155A-23 between isolation valves 146A-1 and IE stack 130A and are respectively configured to supply strong salt solution 111A-1, weak acid solution 112A-1 and weak base solution 113A-1 to IE stack 130A at flow rates and pressures determined by associated control signals in the manner described below. In some embodiments, BPED 110A is operated in a “feed and bleed” mode wherein product fractions (sub-streams) of depleted salt stream 111A-2, strong acid stream 112A-2 and strong base stream 113A-2 are bled off (diverted) for use in the generation of alkalinity product 113A-OUT or other purposes. Referring to the lower portion of FIG. 8, output fraction control valves 146A-2 are configured to facilitate feed-and-bleed operations by dividing the three streams leaving IE stack 130A into corresponding recycle and product fractions (sub-streams). Output fraction control valve 146A-21 divides depleted salt stream 111A-2 into a salt recycle fraction 111A-21 and a salt product fraction 111A-22, where salt recycle fraction 111A-21 is directed back to salt holding tank 121A-1 by way of a conduit 155A-41, and salt product fraction 111A-22 is directed to post-production device 170A. Output fraction control valve 146A-22 divides strong acid stream 112A-2 into an acid recycle fraction 112A-21 and an acid product fraction 112A-22, where acid recycle fraction 112A-21 is directed back to acid holding tank 121A-2 by way of conduit 155A-42, and acid product fraction 112A-22 is directed to an acid neutralization device 177A. Output fraction control valve 146A-23 divides strong base stream 113A-2 into a base recycle fraction 113A-21 and a base product fraction 113A-22, where base recycle fraction 113A-21 is directed back to base holding tank 121A-3 by way of conduit 155A-43, and base product fraction 113A-22 is directed to dilution apparatus 172A. Each fraction control valve 146A-21, 146A-22 and 146A-23 is separately controlled by one or more control signals in the manner, whereby, during enabled (feed-and-bleed) operating modes, control circuit 180 independently controls the volumes (amounts) of each recycle fraction 111A-21, 112A-21 and 113A-21 returned to the holding tanks, and the volumes (amounts) of each product fraction 111A-22, 112A-22 and 113A-22 that is “bled out”. In the depicted embodiment, salt product fraction 111A-22 and base product fraction 113A-22 are mixed to form a base/salt product 113A-3 before being supplied to post-production subsystem 170A to generate ocean alkalinity product 113A-OUT. In other embodiments product fractions 111A-22 and 113A-22 can be transmitted separately to post-production subsystem 170A.

BPED 110A includes BPED-based sensor sets S21, S22 and S23 that are operably disposed to collect data from the various solution streams and to transmit the collected data in real time to controller 180. The general location and function of at least some of these BPED-based sensors is indicated in FIG. 8 and will now be described. In one embodiment, an input sensor set S21 includes sensors configured to measure pressure and composition/concentration from the solution streams flowing in conduits 155A-21, 155A-22 and 155A-23 between isolation valves 146A-1 and IE stack 130A. That is, each sensor of input sensor set S21 is configured to measure the pressure and salt/acid/base concentrations in strong salt stream 111A-1, weak acid stream 112A-1 and weak base stream 113A-1 that are respectively flowing in conduits 155A-31, 155A-32 and 155A-33, and configured to generate associated pressure and composition/concentration data including a salt input pressure value, a salt input composition/concentration value, an acid input pressure value, an acid input composition/concentration value, a base input pressure value and a base input composition/concentration value. Similarly, a set of output sensors S22 and a set of flow rate sensors S23 include sensors configured to measure pressure, composition/concentration and flow rate from the weak (depleted) salt solution stream 111A-2, the strong acid solution stream 112A-2 and the strong base stream 113A-2 flowing from IE stack 135A along conduits 155A-31, 155A-32 and 155A-33, respectively. That is, output sensor set S21 is configured to measure salt/acid/base solution pressure and salt/acid/base concentrations on the downstream side of IE stack 135A and to generate associated data including a salt output pressure value, a salt output composition/concentration value, an acid output pressure value, an acid output composition/concentration value, a base output pressure value and a base output composition/concentration value. Flow rate sensors S23 are configured to measure the flow rate of the salt/acid/base solution streams passing through IE stack 135A. All of the above-mentioned values generated by input sensor set S21, output sensor set S22 and flow rate sensor set S23 are transmitted as sensor data to controller 180. Note that flow control sensor set S23 may be located upstream of IE stack 135A.

Referring to the bottom of FIG. 8, in one embodiment post-production devices 170A include a dilution apparatus (ocean alkalinity production device) 172A, an acid neutralization device 177A, and a flow control device 175A. Dilution apparatus 172A can be configured to generate ocean alkalinity product 113A-OUT by processing and testing base/salt product 113A-3. As mentioned above, to avoid endangering sea life, and to maximize the potential benefits to sea life, ocean alkalinity product 113A-OUT can be a well characterized solution including a mixture of the base substance and saltwater that is released (supplied to ocean 50) only after verifying that the base substance is fully dissolved in the solution, and that the mixture has an appropriate pH value. To this end, dilution apparatus 172A can function to both test/verify and, if necessary, perform post-processing of the base/salt product solution generated by BPED 110A before releasing the verified/processed base solution as ocean alkalinity product 113A-OUT. In one embodiment this processing may include reacting base/salt product 113A-3 with air or CO₂ and/or diluted with seawater or another saltwater solution to generate ocean alkalinity product 113A-OUT with a target pH range. In one embodiment, at least a portion of contaminants removed from seawater 111A-IN by pretreatment unit 140A may be supplied to dilution apparatus 172A for addition to ocean alkalinity product 113A-OUT. Flow control device 175 receives ocean alkalinity product 113A-OUT from dilution apparatus 172A and is configured to control the release of ocean alkalinity product 113-OUT at outfall location 50-0 in the manner described herein. Acid neutralization device 177A receives and processes acid product fraction 112A-22 for power generation or commercial purposes. In one embodiment, device 177A is an electrolyzer configured to generate hydrogen gas H₂ that can be processed by a fuel cell (not shown) to generate supplemental low/zero-carbon electricity to further enhance the economically sustainability of OAE system 100A as a carbon offset system (e.g., by way of transmitting the supplemental electricity to power distribution circuit 190A to reduce the amount of external power consumed by BPED 110A). In some embodiments, acid neutralization device 177A can be configured to produce one or more additional gasses, such as chlorine gas Cl₂ and/or oxygen gas O₂, that may be sold to further enhance the economically sustainability of OAE system 100A as a carbon offset system. It may be desirable to treat acid product fraction 112A-22 before undergoing the acid neutralization process. Some possible methods of pretreatment, not pictured, may include filtration, chemical, electrochemical, nanofiltration, ultrafiltration, reverse osmosis, heating, and cooling. In other embodiments, not pictured, acid product fraction 112A-22 can be utilized in a flow battery to produce supplemental low/zero-carbon electricity to help offset input power.

FIG. 9 shows a method for identifying and selecting one or more suitable release frequencies according to an embodiment in which an ocean CDR system includes a sensor network (e.g., referring to FIG. 1, in cases where the ocean CDR system includes one or more of sensors S1 to S3). In this case, the ocean CDR system utilizes its sensor network to identify and select one or more quiet natural (non-natural) variation frequencies that may be used as the ocean CDR system's release frequencies during batch-release operations. In one embodiment, method generally involves establishing a baseline seawater chemistry schedule (blocks 201B and 202B), utilizing the baseline seawater chemistry schedule to perform test releases and to perform sensor network placement (blocks 205B, 206B and 207B), and then utilizing test data collected by the sensor network to select one or more suitable release frequencies (block 209B) that is/are then utilized to perform batch-release ocean CDR system operations in the manner described above (block 210B).

Referring to the top of FIG. 9, the method begins by positioning the ocean-based sensor(s) of the sensor network in initial locations adjacent to the ocean alkalinity deployment site (outfall location) of the ocean CDR system (block 201B). Next, for a period of time (e.g., weeks to months), the sensors are used to measure characteristics of the seawater chemistry that can be used to verify atmospheric CO₂ removal, such as pH, dissolved inorganic carbon (DIC), and total alkalinity (TA), and the data measured in this manner is used to generate a baseline seawater chemistry schedule of natural variations in the measured seawater chemistry (block 202B). Using the example shown in FIG. 1, positioning the sensor network's sensors may include locating sensors S1 to S3 at initial randomly selected distances outfall location and establishing a baseline seawater chemistry schedule may include utilizing data collected by sensors S1 to S3 to generate a model of natural variations in seawater pH, DIC and/or TA (i.e., time-based changes due to ocean models and changes in ocean/atmospheric conditions). An exemplary partial baseline seawater chemistry schedule that may be collected by sensors S1 to S3 during this process is described above with reference to natural seawater pH variation waveform NV (FIG. 5).

Next, the baseline seawater chemistry schedule is utilized to achieve a functional sensor network placement (e.g., to position the network's sensors at locations that facilitate adequate signal-to-noise ratio) and to analyze potential quiet natural variation frequencies for use as base substance release frequencies. In one embodiment, the baseline schedule of natural variations, the location of the alkalinity deployment site, ocean models of the region surrounding the deployment site, and predicted ocean conditions and atmospheric conditions in the near future are utilized to determine acceptable spatial and temporal locations for each sensor in the sensor network (i.e., such that the increase in pH/DIC/pCO₂/TA caused by the released base substance is reliably measured at each sensor location). In some embodiments placement of the sensor network may be further conditional on one or more of the rate of base substance dispersal at a given location/time, model predictions for TA dispersal and CO₂ removal, and other factors that affect the observed change in seawater chemistry measurement values, and how much is due to the base substance release. In one embodiment, the ocean CDR system's base generating device is controlled to release test amounts of ocean alkalinity product at the various quiet natural variation frequencies identified from the baseline seawater chemistry schedule and measuring the resulting seawater chemistry changes at the initial (current) sensor locations, where the release frequency of each test amount corresponds to one of the identified quiet natural variation frequencies (block 204B). In some embodiments the preliminary seawater chemistry measurement data generated by these test amount releases is utilized to analyze spatial and temporal variations in the seawater chemistry parameters in order to verify or (if necessary) modify the position of each sensor of the sensor network (block 205B). That is, the position of one or more sensors of the sensor network may be modified (block 207B) when the preliminary seawater chemistry measurement data indicates that the spatial and temporal location for each repositioned sensor may further improved by the modification, and then the test amount release process is repeated using the modified/current sensor locations (i.e., control returns along the NO branch from decision block 206B to block 204B). When the preliminary seawater chemistry measurement data indicates that sensor positioning is acceptable (YES branch from decision block 206B), control passes to block 209B.

After placement of the sensor network is completed, a suitable ocean alkalinity product release frequency (or frequencies) is selected/established and operation of the ocean CDR system is initiated using the selected release frequency(s). Referring to block 209B, in one embodiment the suitable ocean alkalinity product release frequency(s) is/are selected from the relatively quiet frequency(s) in the natural pH variation power spectrum signal that exhibit superior signal-to-noise ratios in the preliminary seawater chemistry measurement data collected during sensor network placement. In some embodiments, the test releases of base substance are performed using release frequencies that correspond with various quiet natural variation frequencies, and the resulting test seawater chemistry measurement data collected by the sensor network is utilized to identify suitable release frequency(s) (e.g., the release frequency(s) that produce the highest signal-to-noise ratio). Referring to block 210B (bottom of FIG. 9), after one or more suitable release frequencies are determined, the ocean CDR system's controller operates the ocean CDR system's base generating device and ocean alkalinity product outflow control device according to the method described above (i.e., to release base substance in batches that are released on a schedule determined by the one or more selected release frequencies), and control then passes to block 220 (FIG. 4), whereby subsequently generated sensor data is utilized to measure and/or verify the ocean CDR system's contribution to atmospheric CO₂ removal in the manner described above.

In another embodiment (not pictured) the sensor network utilized for MRV is separate from the ocean CDR system, and data collected by the sensor network is analyzed, or connected to a model or simulation, and one or more release frequencies are then transmitted to the ocean CDR system's controller.

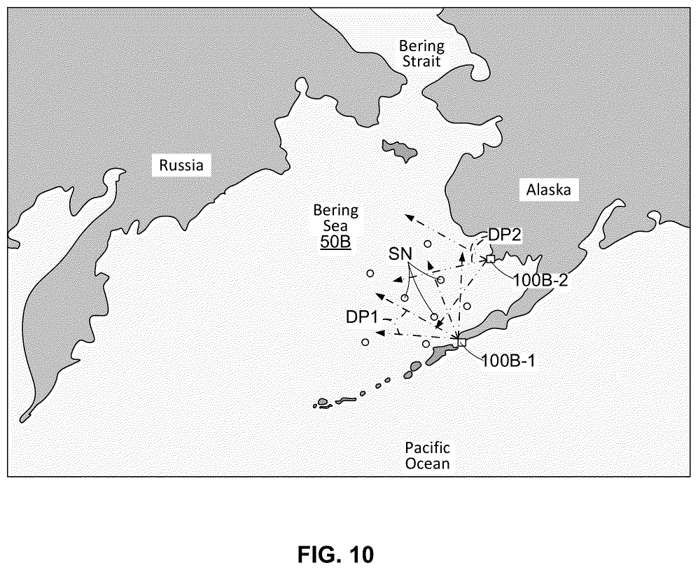

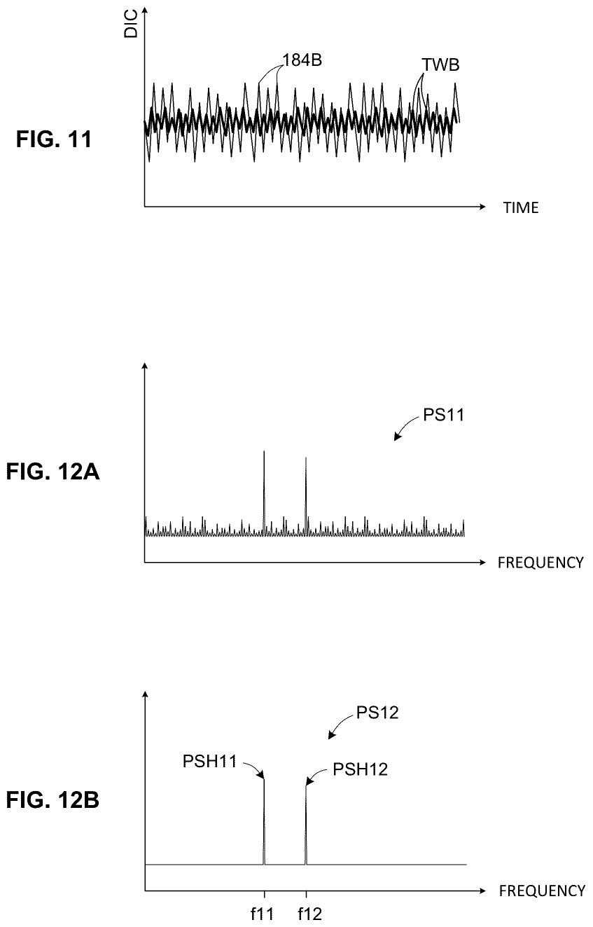

FIGS. 10 to 12B collectively illustrate how a single sensor network may be utilized to simultaneously measure and/or verify contributions from two or more ocean CDR systems according to another exemplary embodiment. FIG. 10 depicts a map depicting an exemplary arrangement including a sensor network SN and two electrochemical OAE (ocean CDR) systems 100-1 and 100-2. FIG. 11 depicts time-based measurement data collected by at least one sensor of sensor network SN during base substance release operations by electrochemical OAE systems 100-1 and 100-2, and FIGS. 12A and 12B show exemplary frequency-based data generated by processing the time-based measurement data of FIG. 11.

Referring to FIG. 10, sensor network SN includes multiple ocean-based sensors (indicated by circles) disposed in the Bering Sea, where all of the sensors are configured to measure one or more seawater chemistry parameters in the manner described above. Both electrochemical OAE systems 100B-1 and 100B-2 are configured to generate and supply ocean alkalinity product into the Bering Sea (ocean) 50B in the manner set forth above with reference to FIG. 1. Electrochemical OAE system 100B-1 is deployed in the Aleutian Islands and electrochemical OAE system 100B-2 is located on mainland Alaska. The depicted example assumes that natural ocean currents disperse the base substances from the associated release points (ocean alkalinity deployment sites) of OAE systems 100B-1 and 100B-2 into the Bering Sea along exemplary dispersion paths DP1 and DP2, respectively, which are indicated by dash-dot and dash-dot-dot arrows, respectively. As indicated, at least some of the ocean-based sensors forming sensor network SN are positioned within expected dispersion paths DP1 and DP2 to facilitate simultaneously detect and/or measure seawater chemistry changes produced by base substance released from both electrochemical OAE systems 100B-1 and 100B-2. In some embodiments, the ocean-based sensors positioned within expected dispersion paths DP1 and DP2 are also utilized to perform baselining prior to the release of ocean alkalinity product in the manner described above with reference to FIG. 9.

In some embodiments, confidence in the seawater chemistry measurement data collected by a single sensor network may be enhanced by controlling two or more ocean CDR systems to release base substance at unique (different) release frequencies. That is, assuming both electrochemical OAE systems 100B-1 and 100B-2 supply the same base substance (e.g., NaOH) to ocean 50B at the same frequency, it would be difficult to determine whether the base substance detected by a given ocean-based sensor was supplied from one or both of OAE system 100B-1 and/or OAE system 100B-2. In the exemplary embodiment shown in FIG. 10 and described below with reference to FIGS. 11, 12A and 12B, the base substance release operations performed by electrochemical OAE systems 100-1 and 100-2 is coordinated such that first electrochemical OAE system 100B-1 is configured to release base substance at a first selected release frequency f11, and second electrochemical OAE system 100B-2 is configured to release base substance at a second selected release frequency f12, where frequency f11 is different from frequency f12, and both frequencies f11 and f12 are predetermined quiet natural variation (non-natural) frequencies.

FIG. 11 is a graph depicting exemplary total time-based DIC measurement data 184B collected by one or more of the sensors of sensor network SN (shown in FIG. 10) in the manner described above with reference to FIG. 1. Note again that total time-based DIC measurement data 184B reflects changes to seawater DIC levels caused by both natural variations and human-induced changes (i.e., changes caused by the ocean alkalinity product supplied from electrochemical OAE systems 100B-1 and 100B-2, both shown in FIG. 10). For illustrative purposes a time-based DIC waveform TWB is superimposed onto total time-based DIC measurement data 184B, where time-based DIC waveform TWB reflects the combined changes to seawater DIC caused by the time-based release of base material from both electrochemical OAE systems 100B-1 and 100B-2.

FIGS. 12A and 12B depict frequency-based data generated by processing time-based DIC measurement data 184B (FIG. 11) in the manner described above. FIG. 12A depicts a power spectrum (frequency-based representation) PS11 that is generated, for example, by utilizing the Fourier transform to convert total time-based DIC measurements of total time-based DIC measurement data 184B into frequency-domain data. FIG. 12B depicts a power spectrum PS12 that is obtained by removing (subtracting or filtering) all previously determined natural variation frequency components from frequency-based representation PS11 (shown in FIG. 12A). In one embodiment, the previously determined natural variation frequency components are obtained from natural seawater chemistry variation data (e.g., natural PH variation power spectrum PSN, described above with reference to FIG. 5), where the natural seawater chemistry variation data was generated and utilized to identify/select frequencies f11 and f12 as the release frequencies for electrochemical OAE systems 100B-1 and 100B-2, respectively. Note that the human-induced spike (power spectra value) PSH11 generated at frequency f11 verifies the significant human-induced contribution provided by first electrochemical OAE system 110B-1, and that human-induced spike PSH12 generated at frequency f12 verifies the significant human-induced contribution provided by second electrochemical OAE system 100B-2. Note also that the subtraction of natural variation frequency components from frequency-based representations may be utilized when verifying/measuring the contributions of a single ocean CDR system.

Although the base release from each of OAE systems 100B-1 and 100B-2 is depicted in FIG. 12B as involving a single release frequency (i.e., base release from OAE system 100B-1 is indicated by power spectra peak PSH11 and base release from OAE system 100B-2 is indicated by power spectra peak PSH12), in practical applications the time waveforms describing the base release from one or both ocean CDR systems may be based on a combination of two or more non-natural frequencies. In such cases, a power spectrum (not shown) would include multiple human-induced spikes related to each of the two or more non-natural frequencies. Alternatively, even if the base substance is supplied from a single ocean CDR system using a single release frequency, distortion and dispersion of the sequentially released batches of ocean alkalinity product may produce multiple human-induced spikes or broader spikes covering a narrow range of frequencies (i.e., when time-based seawater chemistry measurement data collected by remotely located sensors is converted into a corresponding frequency-based representation). However, the inventors believe the main effect caused by diffusion of each released batch is dispersion, and that the released frequency bandwidth (i.e., the range of human-induced spikes) would likely be centered at the single release frequency, whereby the ocean CDR system's contribution may be verified by further processing the multiple human-induced spikes to determine a central frequency. Accordingly, sensor network SN (shown in FIG. 10) performs the function of verifying the contributions of two or more ocean CDR systems. In this way, the present invention provides a reliable Measurement, Reporting, and Verification (MRV) scheme for measuring/verifying atmospheric CO₂ removal produced by a wide range of ocean CDR systems.

Referring again to FIG. 10, in some embodiments, confidence in the seawater chemistry measurement data collected by sensor network SN may also be enhanced by positioning one or more remote ocean-based sensors of sensor network SN outside of expected dispersion paths DP1 and DP2. Because seawater chemistry measurement data collected by such remote ocean-based sensors is solely influenced by natural seawater chemistry variations (i.e., not by the base substance released from OAE systems 100B-1 and/or 100B-2), this seawater chemistry measurement data can be used to determine natural variation frequencies in real-time, and this real-time data may be used in the process described above (i.e., when subtracting natural frequencies from converted seawater chemistry measurement data, as described above with reference to FIG. 12B) to produce reliable and accurate measurement data associated with the base substance released from OAE systems 100B-1 and 100B-2). This approach may also be used to increase confidence in the accuracy of base substance measurements from a single ocean CDR system.

Although the present invention has been described with respect to certain specific embodiments, it will be clear to those skilled in the art that the inventive features of the present invention are applicable to other embodiments as well, all of which are intended to fall within the scope of the present invention. For example, although the invention is primarily described above with specific reference to electrochemical OAE systems, the frequency-based base substance detection methods described above may be utilized in association with any ocean CDR system that, as one of its process steps, generates an ocean alkalinity product including a base substance and utilizes a flow control device (and associated controller) to release the ocean alkalinity product into the ocean.

1. A method for reliably verifying an ocean Carbon Dioxide Removal (ocean CDR) system's contribution to atmospheric CO₂ removal, the ocean CDR system being configured to generate and release an ocean alkalinity product into an ocean at an outfall location such that a base substance included in the ocean alkalinity product disperses into the ocean's seawater along a dispersion path away from the outfall location, the method comprising:
controlling the ocean CDR system to release the ocean alkalinity product as a series of discrete released batches, wherein each said released batch includes an amount of the base substance, and wherein the discrete released batches are sequentially released in accordance with a selected release frequency;
collecting time-based seawater carbonate chemistry measurement data from at least one region of the ocean along the dispersion path;
processing the seawater carbonate chemistry measurement data using frequency-domain techniques to generate a seawater carbonate chemistry variation power spectrum including a plurality of power spectra values for a plurality of frequencies, wherein each power spectrum value indicates a relative contribution to seawater carbonate chemistry occurring at a corresponding frequency of the plurality of frequencies; and
utilizing a human-induced power spectra value of the plurality of power spectra values to verify the release of ocean alkalinity product in said discrete released batches at said selected release frequency.

2. The method of claim 1, further comprising utilizing natural seawater chemistry variation data to identify said selected release frequency.

3. The method of claim 2, wherein utilizing said natural seawater chemistry variation data comprises:
utilizing one or more ocean-based sensors to collect preliminary time-based seawater carbonate chemistry measurement data from the ocean's seawater at one or more locations along the base substance's dispersion path;
processing the preliminary seawater carbonate chemistry measurement data using frequency-domain techniques to generate a natural seawater carbonate chemistry variation power spectrum;
identifying one or more quiet natural variation frequencies in said natural seawater carbonate chemistry variation power spectrum, wherein each of said one or more quite frequencies has an associated insignificant power spectra value; and
utilizing said one or more quiet natural variation frequencies as said selected release frequency.

4. The method of claim 1, wherein the ocean CDR system comprises:
a base generating device configured to generate the ocean alkalinity product; and
a flow control device configured to release the ocean alkalinity product into the ocean's seawater when in an opened control state, and configured to prevent the release of the ocean alkalinity product when in a closed control state,
wherein controlling the ocean CDR system comprises repeatedly cycling the flow control device between the opened control state and the closed control state in accordance with at least one release frequency.

5. The method of claim 1, wherein collecting said time-based seawater carbonate chemistry measurement data comprises utilizing one or more ocean-based sensors, wherein each said ocean-based sensor is disposed in the base substance's dispersion path and located at an associated distance from the outfall location, and each said ocean-based sensor is configured to measure a seawater carbonate chemistry parameter from the ocean's seawater.

6. The method of claim 5, wherein collecting the time-based seawater carbonate chemistry measurement data comprises utilizing the one or more ocean-based sensors to measure one or more of pH, dissolved organic carbon (DIC), partial pressure of CO₂ (PCO₂) and total alkalinity (TA) of the ocean's seawater.

7. The method of claim 1, processing the seawater carbonate chemistry measurement data comprises utilizing Fourier transform conversion to generate the seawater carbonate chemistry variation power spectrum.

8. The method of claim 7, further comprising subtracting a natural PH variation power spectrum from the seawater carbonate chemistry variation power spectrum to identify the human-induced power spectra value.

9. The method of claim 1, further comprising utilizing the human-induced power spectra value to measure the ocean CDR system's contribution to atmospheric CO₂ removal.

10. The method of claim 1, further comprising:
disposing one or more ocean-based sensors at one or more corresponding initial positions along the base substance's dispersion path, and utilizing preliminary time-based seawater carbonate chemistry measurement data collected from the ocean's seawater to establish a baseline seawater chemistry schedule;
releasing test amounts of said base substance at the outfall location in accordance with one or more variation natural frequencies identified in said baseline seawater chemistry schedule; utilizing spatial and temporal variations in preliminary seawater chemistry measurement data collected by the one or more ocean-based sensors in response to said base substance test amount releases to reposition one or more ocean-based sensors from one or more of said corresponding initial positions until the one or more one or more ocean-based sensors are in suitable positions; and
utilizing signal-to-noise ratios in said preliminary seawater chemistry measurement data to identify said selected release frequency.

11. A method for simultaneously verifying contributions to atmospheric CO₂ removal by a first ocean Carbon Dioxide Removal (ocean CDR) system and a second ocean CDR system, the first ocean CDR system being configured to generate and release a first base substance into an ocean such that the first base substance disperses into the ocean's seawater along a first dispersion path, the second ocean CDR system being configured to generate and release a second base substance into the ocean such that the second base substance disperses along a second dispersion path that overlaps with the first dispersion path, wherein the method comprises:
controlling the first and second ocean CDR systems such that first base substance generated by the first ocean CDR system is released as a first series of discrete released batches in accordance with a first release frequency, and such that second base substance generated by the second ocean CDR system is released as a second series of discrete released batches in accordance with a second release frequency, the first release frequency being different from the second release frequency;
collecting time-based seawater chemistry measurement data from one or more ocean-based sensors disposed in the ocean such that the time-based seawater chemistry measurement data is simultaneously influenced by both of said first and second series of release batches; and
processing the seawater chemistry measurement data using frequency-domain techniques to generate frequency-based data including a first power spectra value at the first release frequency and a second power spectra value at the second release frequency; and
utilizing the first and power spectra values to verify the simultaneous contributions to atmospheric CO₂ removal by the first and second ocean CDR systems.

12. The method of claim 11, further comprising utilizing preliminary seawater chemistry measurement data collected by the one or more ocean-based sensors to generate natural seawater chemistry variation data and utilizing the preliminary seawater chemistry measurement data to identify the first and second release frequencies.

13. The method of claim 11,
wherein controlling the first ocean CDR system comprises repeatedly cycling a first flow control device between opened and closed control states in accordance with the first release frequency such that one discrete release batch of said first series of discrete released batches is released into the ocean during each said opened control state of said first flow control device, and
wherein controlling the second ocean CDR system comprises repeatedly cycling a second flow control device between opened and closed control states in accordance with the second release frequency such that one discrete release batch of said second series of discrete released batches is released into the ocean during each said opened control state of said second flow control device.

14. The method of claim 11, wherein collecting the time-based seawater carbonate chemistry measurement data comprises utilizing the one or more ocean-based sensors to measure one or more of pH, dissolved organic carbon (DIC), partial pressure of CO₂ (PCO₂) and total alkalinity (TA) of the ocean's seawater.

15. The method of claim 11, processing the seawater carbonate chemistry measurement data comprises utilizing Fourier transform conversion to generate a seawater carbonate chemistry variation power spectrum.

16. The method of claim 15, further comprising subtracting a natural PH variation power spectrum from the seawater carbonate chemistry variation power spectrum to identify the first and second power spectra values.

17. An ocean Carbon Dioxide Removal (ocean CDR) system comprising:
a base generating device configured to generate an ocean alkalinity product including a base substance;
a flow control device configured to control a volumetric release rate of the ocean alkalinity product into the ocean at an outfall location; and
a controller configured to control operation of the flow control device such that the volumetric release rate of the ocean alkalinity product varies in accordance with a selected release frequency such that the ocean alkalinity product is released into the ocean as a series of release batches,
wherein the selected release frequency coincides with a quiet natural seawater carbonate chemistry variation frequency such that the base substance in the released ocean alkalinity product dispersed into the ocean's seawater along an associated dispersion path away from the outfall location produces a time waveform whose power spectrum contains significant contributions at the quiet natural seawater carbonate chemistry variation frequency.

18. The ocean CDR system of claim 17, further comprising one or more ocean-based sensors respectively disposed at associated sensor deployment locations along the associated dispersion path, wherein each of the one or more sensors is configured to collect time-based seawater carbonate chemistry measurement data from said associated sensor deployment location.

19. The ocean CDR system of claim 18, wherein the controller is configured to receive said time-based seawater carbonate chemistry measurement data from the one or more ocean-based sensors, and is configured to verify the contribution of the ocean CDR system to atmospheric CO₂ removal by:
processing the seawater carbonate chemistry measurement data using frequency-domain techniques to generate a seawater carbonate chemistry variation power spectrum including a plurality of power spectra values for a plurality of frequencies, wherein each power spectrum value indicates a relative contribution to seawater carbonate chemistry occurring at a corresponding frequency of the plurality of frequencies; and
utilizing a human-induced power spectra value of the plurality of power spectra values to verify the release of ocean alkalinity product in said discrete released batches at said selected release frequency.

20. The ocean CDR system of claim 18, wherein the one or more ocean-based sensors are configured to measure one or more of pH, dissolved organic carbon (DIC), partial pressure of CO₂ (pCO₂) and total alkalinity (TA) of the ocean's seawater.
